(19) United States
(12) Patent Application Publication
(10) Pub. No.: US 2009/0209602 A1
(43) Pub. Date: Aug. 20, 2009

(54) CHEMICAL COMPOUNDS

(76) Inventors: Roger John Butlin, Cheshire (GB); Robert Davies, Cheshire (GB); William McCoull, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 12/303,521

(22) PCT Filed: Jun. 4, 2007
(86) PCT No.: PCT/GB2007/002048

§ 371 Date: Dec. 4, 2008

Related U.S. Application Data
(60) Provisional application No. 60/811,375, filed on Jun. 6, 2006.

(51) Int. Cl. A61K 31/421; C07D 263/48; A61P 3/10

(52) U.S. Cl. 514/377; 548/234

(57) ABSTRACT

Compounds of formula (I), or salts thereof, which inhibit acetyl CoA(acetyl coenzyme A):diacylglycerol acyltransferase (DGAT1) activity are provided,

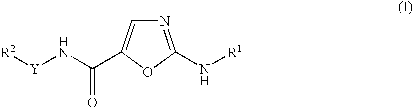

wherein, for example, R¹ is an optionally substituted aryl or optionally substituted heteroaryl group, Y is a direct bond, or a defined linking group and R² is an optionally substituted aryl, an optionally substituted cycloalkyl or an optionally substituted heterocyclic group; together with processes for their preparation, pharmaceutical compositions containing them and their use as medicaments.

The present invention relates to compounds which inhibit acetyl CoA(acetyl coenzyme A):diacylglycerol acyltransferase (DGAT1) activity, processes for their preparation, pharmaceutical compositions containing them as the active ingredient, methods for the treatment of disease states associated with DGAT1 activity, to their use as medicaments and to their use in the manufacture of medicaments for use in the inhibition of DGAT1 in warm-blooded animals such as humans. In particular this invention relates to compounds useful for the treatment of type II diabetes, insulin resistance, impaired glucose tolerance and obesity in warm-blooded animals such as humans, more particularly to the use of these compounds in the manufacture of medicaments for use in the treatment of type II diabetes, insulin resistance, impaired glucose tolerance and obesity in warm-blooded animals such as humans.

Acyl CoA:diacylglycerol acyltransferase (DGAT) is found in the microsomal fraction of cells. It catalyzes the final reaction in the glycerol phosphate pathway, considered to be the main pathway of triglyceride synthesis in cells by facilitating the joining of a diacylglycerol with a fatty acyl CoA, resulting in the formation of triglyceride. Although it is unclear whether DGAT is rate-limiting for triglyceride synthesis, it catalyzes the only step in the pathway that is committed to producing this type of molecule [Lehner & Kuksis (1996) Biosynthesis of triacylglycerols. Prog. Lipid Res. 35: 169-201].

Two DGAT genes have been cloned and characterised. Both of the encoded proteins catalyse the same reaction although they share no sequence homology. The DGAT1 gene was identified from sequence database searches because of its similarity to acyl CoA:cholesterol acyltransferase (ACAT) genes. [Cases et al (1998) Identification of a gene encoding an acyl CoA:diacylglycerol acyltransferase, a key enzyme in triacylglycerol synthesis. Proc. Natl. Acad. Sci. USA 95: 13018-13023]. DGAT1 activity has been found in many mammalian tissues, including adipocytes.

Because of the previous lack of molecular probes, little is known about the regulation of DGAT1. DGAT1 is known to be significantly up-regulated during adipocyte differentiation.

Studies in gene knockout mice has indicated that modulators of the activity of DGAT1 would be of value in the treatment of type II diabetes and obesity. DGAT1 knockout (Dgat1^{-/-}) mice, are viable and capable of synthesizing triglycerides, as evidenced by normal fasting serum triglyceride levels and normal adipose tissue composition. Dgat1^{-/-} mice have less adipose tissue than wild-type mice at baseline and are resistant to diet-induced obesity. Metabolic rate is ˜20% higher in Dgat1^{-/-} mice than in wild-type mice on both regular and high-fat diets [Smith et al (2000) Obesity resistance and multiple mechanisms of triglyceride synthesis in mice lacking DGAT. Nature Genetics 25: 87-90]. Increased physical activity in Dgat1^{-/-} mice partially accounts for their increased energy expenditure. The Dgat1^{-/-} mice also exhibit increased insulin sensitivity and a 20% increase in glucose disposal rate. Leptin levels are 50% decreased in the Dgat1^{-/-} mice in line with the 50% decrease in fat mass.

When Dgat1^{-/-} mice are crossed with ob/ob mice, these mice exhibit the ob/ob phenotype [Chen et al (2002) Increased insulin and leptin sensitivity in mice lacking acyl CoA:diacylglycerol acyltransferase J. Clin. Invest. 109:1049-1055] indicating that the Dgat1^{-/-} phenotype requires an intact leptin pathway. When Dgat1^{-/-} mice are crossed with Agouti mice a decrease in body weight is seen with normal glucose levels and 70% reduced insulin levels compared to wild type, agouti or ob/ob/ Dgat1^{-/-} mice.

Transplantation of adipose tissue from Dgat1^{-/-} mice to wild type mice confers resistance to diet-induced obesity and improved glucose metabolism in these mice [Chen et al (2003) Obesity resistance and enhanced glucose metabolism in mice transplanted with white adipose tissue lacking acyl CoA:diacylglycerol acyltransferase J. Clin. Invest. 111: 1715-1722].

International Patent Applications WO2004/047755 (Tularik and Japan Tobacco) and WO2005/013907 (Japan Tobacco and Amgen) describe fused bicyclic nitrogen-containing heterocycles which are inhibitors of DGAT-1. JP2004-67635 (Otsuka Pharmaceuticals) describes thiazoleamido substituted phenyl compounds which are further substituted with alkylphosphonates and which inhibit DGAT-1. WO2004/100881 (Bayer) describes biphenylamino compounds substituted with imidazole, oxazole or thiazole which inhibit DGAT-1. Our co-pending International Application PCT/GB2005/004726 describes oxadiazole compounds which inhibit DGAT-1.

Accordingly, the present invention provides a compound of formula (I)

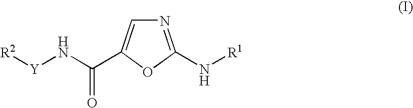

or a salt thereof, wherein:

R¹ is an optionally substituted aryl or optionally substituted heteroaryl group, wherein the optional substituents are one or more groups selected from a group -Z^{a}, a group —X²—(CR⁵²R⁵³)_{w}-Z^{a}, a group —X²—(CR⁵²R⁵³)ₐ—X³-Z^{a}, a group —(CR⁵²R⁵³)ₐ—X³-Z^{a} or a functional group (which is other than a group —X²—(CR⁵²R⁵³)_{w}-Z^{a} or a group —X²—(CR⁵²R⁵³)ₐ—X³-Z^{a});

Y is a direct bond, or a group (CR⁴⁰R⁴¹)ₛ or —X⁶ (CR⁴⁰R⁴¹)ₜ— where each R⁴⁰ and R⁴¹ is independently selected from hydrogen, (1-4C)alkyl, hydroxy, halo, halo(1-4C)alkyl, amino, cyano, (1-4C)alkoxy, (1-4C)haloalkoxy or ((1-3C)alkyl)CONH—, s is an integer of from 1 to 6 and t is an integer of from 1 to 6, provided that the X⁶ atom of the group —X⁶(CR⁴⁰R⁴¹)ₜ— is attached to the R² group and that a single sp³ hybridised carbon atom does not carry two or more bonds to a heteroatom unless the heteratom is a halo;

R² is an optionally substituted aryl, an optionally substituted cycloalkyl or an optionally substituted heterocyclic group, wherein optional substitutents are one or more groups selected from a group -Z, a group —X—(CR⁴²R⁴³)ᵤ-Z, a group —X—(CR⁴²R⁴³)ᵥ—X¹-Z or a group —CR⁴²R⁴³)ᵥX¹-Z or a functional group (which is other than a group —X—(CR⁴²R⁴³)ᵤ-Z or a group —X—(CR⁴²R⁴³)ᵥ—X¹-Z);

wherein Z and Z^{a} are independently selected from a hydrocarbyl group or a heterocyclic group or a combination thereof, wherein the group Z and Z^{a} is optionally substituted on any available atom by one or more functional groups, or by a group —X⁷—(CR⁶²R⁶³)_{b}R⁶⁴;

X, X¹, X², X³, X⁶ and X⁷ are linking groups independently selected from —C(O)ₓ—, —O—, —S(O)_{y}—, —NR⁴⁴—, —C(O)NR⁴⁴—, —OC(O)NR⁴⁴—, —CH═NO—, —NR⁴⁴C(O)ₓ—, —NR⁴⁴CONR⁴⁵—, —S(O)₂NR⁴⁴— or —NR⁴⁴S(O)₂— where x is an integer of 1 or 2, y is 0, 1 or 2, and R⁴⁴ and R⁴⁵ are independently selected from hydrogen or (1-6C)alkyl,
u and w are independently selected from 0 or an integer of from 1 to 6;
v, a and b are independently selected from an integer of from 1 to 6;
each R⁴², R⁴³, R⁵², R⁵³, R⁶² and R⁶³ is independently selected from hydrogen, (1-4C)alkyl, hydroxy, halo, halo(1-4C)alkyl, amino, cyano, (1-4C)alkoxy, (1-4C)haloalkoxy, ((1-3C) alkyl)CONH—, carboxy or a carboxylic acid mimic or bioisostere thereof, and R⁶⁴ is a functional group.

As used herein, the term “functional group” includes carboxy, halo, halo(1-6C)alkyl, cyano, nitro, —C(O)R²⁰, a carboxylic acid mimic or bioisostere thereof, —OR²⁰, S(O)ₘR²⁰, —OS(O)R²⁰, —NR²¹R²², —C(O)NR²¹R²², —OC(O)NR²¹R²², —CH═NOR²⁰, —NR²¹C(O)R²⁰, —NR²⁰CONR²¹R²², —N═CR²¹R²², —S(O)₂NR²¹R²² or —NR²S(O)₂R²²; where R²⁰, R²¹ and R²² are independently selected from hydrogen or optionally substituted hydrocarbyl or optionally substituted heterocyclyl, or R²¹ and R²² together with the nitrogen atom to which they are attached form an optionally substituted ring having from 3 to 10 atoms, which optionally contains further heteroatoms such as S(O)ₘ, oxygen and nitrogen, n is an integer of 1 or 2, m is 0 or an integer of 1-2.

Suitable optional substituents for hydrocarbyl groups or heterocyclic groups R²⁰, R²¹ and R²² (including for rings formed by NR²¹R²²) include halo, halo(1-4C)alkyl (such as trifluoromethyl, difluoromethyl or fluoromethyl), mercapto, hydroxy, alkoxy, oxo, heteroaryloxy, alkenyloxy, alkynyloxy, alkoxyalkoxy, aryloxy (where the aryl group may be substituted by halo, cyano, nitro, hydroxy(1-4C)alkyl, halo(1-4C)alkyl, amino, (1-4C)alkoxy, (1-4C)haloalkoxy, ((1-3C)alkyl)CONH—, carboxy or a carboxylic acid mimic or bioisostere thereof), cyano, nitro, amino, mono- or di-alkyl amino, alkylamido, oximino (for example hydroxyimino or alkyloxyimino), carbamoyl, carboxy or a carboxylic acid mimic or bioisostere thereof, or —S(O)ₘR²³ where m is as defined above and R²³ is alkyl (optionally substituted by one or more groups selected from hydroxy, halo, amino, cyano, ((1-3C)alkyl)CONH—, carboxy or a carboxylic acid mimic or bioisostere thereof), (1-6C)alkoxy, (1-6C)alkoxycarbonyl, carbamoyl, N-((1-6C)alkyl)carbamoyl, halo(1-6C)alkyl (such as trifluoromethyl), (1-6C)alkylsulphonyl, (1-6C)alkylsulphinyl. Heterocyclic groups R²⁰, R²¹ and R²² may also be optionally substituted by one or more hydrocarbyl groups such as (1-4C)alkyl.

In this specification the term “alkyl” includes both straight and branched chain alkyl groups but references to individual alkyl groups such as “propyl” are specific for the straight chain version only. An analogous convention applies to other generic terms. Unless otherwise stated the term “alkyl” advantageously refers to chains with 1-10 carbon atoms, suitably from 1-6 carbon atoms, preferably 1-4 carbon atoms.

In this specification the term “alkoxy” means an alkyl group as defined hereinbefore linked to an oxygen atom.

It is to be understood that optional substituents on any group may be attached to any available atom as appropriate unless otherwise specified, including heteroatoms provided that they are not thereby quaternised.

In this specification the term “heteroatom” refers to non-carbon atoms such as oxygen, nitrogen or sulphur atoms. In addition, where the heteroatom may have a single valency, it may comprise a halo. The terms “alkenyl” and “alkynyl” refer to unsaturated straight or branched structures, which unless specified otherwise, contain for example from 2 to 10, preferably from 2 to 6 carbon atoms. Cyclic moieties such as cycloalkyl and cycloalkenyl are similar in nature but have at least 3 carbon atoms. Examples of alkyl, alkenyl and cycloalkyl groups are given hereinafter, such as examples of (1-6C)alkyl, (3-8C)cycloalkyl etc.

References to aryl groups include aromatic carbocylic groups such as phenyl and naphthyl, as well as partially aromatic groups such as indenyl and indanyl. The term “aralkyl” refers to aryl substituted alkyl groups such as benzyl.

The term “heterocyclyl” or “heterocyclic” includes saturated or unsaturated rings, which may be aromatic, non-aromatic rings or partially aromatic, for example containing from 3 to 20, suitably from 4 to 10 ring atoms, at least one of which is a heteroatom such as oxygen, sulphur or nitrogen. They may be mono- or bicyclic ring systems, wherein one or both rings may be saturated or unsaturated, for example they may be aromatic. In particular, bicyclic ring systems will comprise fused 5,6-membered or 6,6-membered rings.

“Heteroaryl” refers to those heterocyclic groups described above which have an aromatic character. Where “heteroaryl” is a bi-cyclic ring system, then at least one ring is aromatic and one or both rings contain ring heteroatoms.

In general, heteroaryl examples of monocyclic heterocyclyl rings include furyl, thienyl, pyrrolyl, imidazolyl, triazolyl, thiazolyl, tetrazolyl, oxazolyl, isoxazolyl, pyrazolyl, pyridyl, pyrimidinyl, pyrazinyl, pyridazinyl and triazinyl.

Examples of non-heteroaryl monocyclic heterocyclic rings include morpholino, thiomorpholino (and versions thereof wherein the sulfur is oxidised), pyrrolidinyl, tetrahydrofuryl, tetrahydrothienyl, piperazinyl and piperidinyl, 2-oxopiperidinyl, 2-oxopyrrolidinyl, tetrahydropyranyl, dihydropyranyl, azetidinyl, homomorpholinyl, diazepinyl and azepinyl.

Suitable examples of bicyclic heteroaryl rings include indolyl, quinolinyl, isoquinolinyl, quinoxalinyl, benzthiazolyl, benzoxazolyl, benzothienyl, benzofuryl, benzimidazolyl, benzodioxolanyl, pyrrolopyridyl, quinazolinyl, purinyl, and naphthyridinyl. It will be understood that structures such as 2-oxo-2,3-dihydro-1H-benzimidazolyl and oxothiadiazolyl which fall within the definition of the term heteroaryl, retain their aromatic characteristics in both rings by virtue of tautomerism. Suitable examples of bicyclic heterocyclic rings include 1,3-benzodioxol-5-yl, chromanyl and isochromanyl.

Other expressions used in the specification include “hydrocarbyl” which refers to any structure comprising carbon and hydrogen atoms. These may be arranged in rings or chains or combinations in which rings are joined to chains or to further rings, or a fused to further rings. Generally, hydrocarbyl groups will contain from 1 to 20, for instance from 1-12 carbon atoms. These may be alkyl, alkenyl, alkynyl, aryl, aralkyl, aralkenyl, aralkynyl, cycloalkyl or cycloalkenyl, wherein any cyclic moiety such as aryl, aralkyl, cycloalkyl or cycloalkenyl are optionally substituted with alkyl, alkenyl, alkynyl and/or with further cyclic moieties, and where any alkyl, alkenyl or alkynyl groups are optionally substituted with cycloalkyl, or cycloalkenyl. The term cycloalkyl also includes bi- and tri-cycloalkyl rings, such as adamantyl and bicyclo[2.2.2]octanyl.

Suitable combinations of rings and chains which are comprised by the term hydrocarbyl include

a) cycloalkyl linked to a (1-6C)alkyl group (in particular cyclohexylmethyl, cyclopentylmethyl, cyclobutylmethyl, cyclohexylethyl), or to two (1-6C)alkyl groups (for example methylcyclobutylmethyl);

b) cyclohexyl linked to a second cyclohexyl or a cyclopentyl group by a direct bond, or with a (1-6C)alkyl group linker;

c) a phenyl group linked to a second phenyl group by a direct bond, or with a (1-6C)alkyl group linker;

d) a (3-8C)cycloalkyl group (such as cyclohexyl or cyclopentyl) linked to a phenyl group by a direct bond or with a (1-6C)alkyl linker;

e) a benzyl or methylphenyl (such as tolyl) group.

References to a “combination” of hydrocarbyl and heterocyclic groups refer to moieties which contain one or more heterocyclic groups joined together, or one or more heterocyclic groups joined to one or more hydrocarbyl groups.

Suitable combinations of hydrocarbyl and heterocyclic groups include a heterocyclyl group (such as pyridyl, morpholino, thiomorpholino, piperazinyl or piperidinyl) linked to (or substituted by) a hydrocarbyl group (such as a (1-6C)alkyl group and/or a (3-8C)cycloalkyl group; in particular a (1-6C)alkyl group). For example methylpyridyl (wherein the methyl may be further substituted by a functional group such as carboxy), benzylpiperazine, (methyl)oxopyridazine, (methyl)oxothiadiazole, (optionally carboxy substituted)methylpiperidyl, (optionally carboxy substituted)methylpiperidylmethyl, (optionally carboxy substituted)dimethylpiperidyl, (optionally carboxy substituted)ethylpiperidyl and (cyclopropylmethyl)piperazinyl.

Unless specified otherwise, the expression “haloalkyl” refers to alkyl groups which carry at least one halo substitutent. This includes perhalo groups where all hydrogen atoms are replaced by halo such as fluoro.

It is to be understood that optional substituents on any group may be attached to any available atom as appropriate unless otherwise specified, including heteroatoms provided that they are not thereby quaternised.

Within this specification composite terms are used to describe groups comprising more than one functionality such as -(1-6C)alkylNHSO₂(1-6C)alkyl. Such terms are to be interpreted in accordance with the meaning which is understood by a person skilled in the art for each component part. For example -(1-6)alkylNHSO₂(1-6C)alkyl includes -methylaminosulfonylmethyl, -methylaminosulfonylethyl, -ethylaminosulfonylmethyl, and -propylaminosulfonylbutyl.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chosen from “0, 1 or 2” groups and “1 or 2” and any other analogous groups.

Substituents may be present at any suitable position on, for example, an alkyl group. Therefore, hydroxy substituted (1-6C)alkyl includes hydroxymethyl, 1-hydroxyethyl, 2-hydroxyethyl and 3-hydroxypropyl.

Examples of (1-4C)alkyl include methyl, ethyl, propyl and isopropyl; examples of (1-6C)alkyl include methyl, ethyl, propyl, isopropyl, t-butyl, pentyl, iso-pentyl, 1-2-dimethylpropyl and hexyl; examples of (2-6C)alkenyl include ethenyl, propenyl, isopropenyl, 2-pentenyl, 3-pentenyl, 4-pentenyl, 2-methylpropenyl and hexenyl; examples of (2-6C)alkynyl include ethynyl, propynyl, 2-pentynyl, 3-pentynyl, 4-pentynyl and hexynyl; examples of (1-4C)alkoxy include methoxy, ethoxy, propoxy, isopropoxy and tert-butoxy; examples of (1-6C)alkoxy include methoxy, ethoxy, propoxy, isopropoxy, tert-butoxy and pentoxy; examples of (1-6C)alkoxy(1-6C)alkyl include methoxymethyl, ethoxymethyl, methoxyethyl, propoxymethyl, isopropoxymethyl, pentoxyethyl, methoxyhexyl and tert-butoxybutyl; examples of (3-8C)cycloalkyl include (3-6C)cycloalkyl (cyclopropyl, cyclobutyl, cyclopentyl, cyclohexyl), cycloheptyl and cycloctyl; examples of (3-8C)cycloalkoxy include cyclopropoxy, cyclobutoxy, cyclopentoxy, cyclohexyloxy, cyclopentyloxy and cyclooctyloxy; examples of (3-8C)cycloalkyl(1-6C)alkyl include cyclopropylmethyl, cyclopropylethyl, cyclopropylbutyl, cyclobutylmethyl, cyclopentylmethyl, cyclohexylmethyl, cyclopentylethyl and cyclooctylpropyl; examples of (3-8C)cycloalkoxy(1-6C)alkyl include cyclopropoxymethyl, cyclopropoxyethyl, cyclopropoxybutyl, cyclobutoxymethyl, cyclopentoxymethyl, cyclohexyloxymethyl, cyclopentoxyethyl and cyclooctyloxypropyl; examples of (3-8C)cycloalkoxy(1-6C)alkoxy include cyclopropoxymethoxy, cyclopropoxyethoxy, cyclopropoxybutoxy, cyclobutoxymethoxy, cyclopentoxymethoxy, cyclohexyloxymethoxy cyclopentoxyethoxy and cyclooctyloxypropoxy; examples of (3-8C)cycloalkoxy(1-6C)alkoxy(1-6C)alkyl include cyclopropoxymethoxymethyl, cyclopropoxyethoxymethyl, cyclopropoxybutoxymethyl, cyclobutoxymethoxyethyl, cyclopentoxymethoxypropyl, cyclohexyloxymethoxymethyl cyclopentoxyethoxymethyl and cyclooctyloxypropoxymethyl; examples of halo are chloro, bromo, iodo and fluoro; examples of halo(1-6C)alkyl include halo(1-4C)alkyl such as chloromethyl, fluoroethyl, fluoromethyl, fluoropropyl, fluorobutyl, dichloromethyl, difluoromethyl, 1,2-difluoroethyl and 1,1-difluoroethyl as well as perhalo(1-6C)alkyl (including perhalo(1-4C)alkyl) such as trifluoromethyl, pentafluoroethyl, and heptafluoropropyl; examples of halo(1-6C)alkoxy include halo(1-4C)alkoxy such as chloromethoxy, fluoroethoxy and fluoromethoxy, difluoromethoxy, as well as perhaloalkoxy such as pentafluoroethoxy, trifluoromethoxy and heptafluoropropoxy; examples of hydroxy(1-6C)alkyl include hydroxy(1-4C)alkyl such as hydroxy methyl, 1-hydroxyethyl, 2-hydroxyethyl and 3-hydroxybutyl; example of carboxy(1-6C)alkyl include carboxy(1-4C)alkyl, such as carboxymethyl, carboxyethyl, carboxypropyl and carboxybutyl; examples of amino(1-6C)alkyl include aminomethyl, aminoethyl, 2-aminopropyl, 3-aminopropyl, 2-aminoiso-propyl, aminobutyl and 2-aminotert-butyl; examples of (1-6C)alkylamino include (1-4C)alkylamino such as methylamino, ethylamino and propylamino; examples of di-((1-6C)alkyl)amino include di-(1-4C)alkylamino such as dimethylamino, N-ethyl-N-methylamino, diethylamino, N-methyl-N-propylamino and di-isopropylamino; examples of (1-6C)alkylcarbonyl include (1-4C)alkylcarbonyl such as methylcarbonyl, ethylcarbonyl, propylcarbonyl, iso-propylcarbonyl and tert-butylcarbonyl; examples of (1-6C)alkylcarbonyloxy include (1-4C)alkylcarbonyloxy such as methylcarbonyloxy, ethylcarbonyloxy, propylcarbonyloxy, iso-propylcarbonyloxy and tert-butylcarbonyloxy; examples of (1-6C)alkoxycarbonyl (N-(1-6C)alkylcarbamoyl) include (1-4C)alkoxycarbonyl such as methoxycarbonyl, ethoxycarbonyl, propoxycarbonyl, iso-propoxycarbonyl and tert-butoxycarbonyl; examples of (1-6C)alkoxycarbonylamino include (1-4C)alkoxycarbonylamino such as methoxycarbonylamino, ethoxycarbonylamino, propoxycarbonylamino, iso-propoxycarbonylamino and tert-butoxycarbonylamino; examples of (1-6C)alkoxycarbonyl(N-methyl)amino include (1-4C)alkoxycarbonyl(N-methyl)amino such as methoxycarbonyl(N-methyl)amino, ethoxycarbonyl(N-methyl)amino, propoxycarbonyl(N-methyl)amino, iso-propoxycarbonyl(N-methyl)amino and tert-butoxycarbonyl(N-methyl)amino; examples of (1-6C)alkylthio include methylthio, ethylthio, propylthio, isopropylthio and butylthio; examples of (1-6C)alkylsulfinyl include methylsulfinyl, ethylsulfinyl, propylsulfinyl, isopropylsulfinyl and butylsulfinyl; examples of (1-6C)alkylsulfonyl include methylsulfonyl, ethylsulfonyl, propylsulfonyl, isopropylsulfonyl and butylsulfonyl; examples of (1-6C)alkoxysulfonyl include methoxysulfonyl, ethoxysulfonyl, propoxysulfonyl, isopropoxysulfonyl and butoxysulfonyl; examples of (1-6C)alkylcarbonylamino include (1-4C)alkylcarbonylamino such as (1-3C)alkylCONH) (methylcarbonylamino, ethylcarbonylamino, propylcarbonylamino, iso-propylcarbonylamino) and tert-butylcarbonylamino; examples of (1-6C)alkylaminocarbonyl include (1-4C)alkylaminocarbonyl such as methylaminocarbonyl, ethylaminocarbonyl, propylaminocarbonyl, iso-propylaminocarbonyl and tert-butylaminocarbonyl; examples of di(1-6C)alkylaminocarbonyl include di(1-4C)alkylaminocarbonyl such as dimethylaminocarbonyl, N-methyl-N-ethylaminocarbonyl, diethylaminocarbonyl, N-methyl-N-propylaminocarbonyl and di-isopropylaminocarbonyl; examples of (1-6C)alkylaminocarbonyloxy include (1-4C)alkylaminocarbonyloxy such as methylaminocarbonyloxy, ethylaminocarbonyloxy, propylaminocarbonyloxy, iso-propylaminocarbonyloxy and tert-butylaminocarbonyloxy; examples of —S(O)ₚ(1-4C)alkyl (wherein p is 0, 1 or 2) include (1-6C)alkylthio, (1-6C)alkylsulfinyl and (1-6C)alkylsulfonyl; examples of (1-6C)alkylaminosulfonyl include —SO₂NH(1-4C)alkyl such as methylaminosulfonyl, ethylaminosulfonyl, propylaminosulfonyl, iso-propylaminosulfonyl and tert-butylaminosulfonyl; examples of di(1-6C)alkylaminosulfonyl include di(1-4C)alkylaminosulfonyl such as dimethylaminosulfonyl, N-methyl-N-ethylaminosulfonyl, diethylaminosulfonyl, N-methyl-N-propylaminosulfonyl and di-isopropylaminosulfonyl; examples of (1-6C)alkylsulfonylamino include (1-4C)alkylsulfonylamino such as methylsulfonylamino, ethylsulfonylamino, propylsulfonylamino, iso-propylsulfonylamino and tert-butylsulfonylamino.

Particular values of variable groups in compounds of formula (I) are as follows. Such values may be used where appropriate with any of the other values, definitions, aspects, claims or embodiments defined hereinbefore or hereinafter.

In a particular embodiment, R¹ is an optionally substituted aryl group such as optionally substituted phenyl or napthyl. R¹ as an optionally substituted aryl group may also be indanyl. It will be understood that when R¹ is a partially saturated aryl group, such as indanyl, it is the aromatic ring portion of R¹ which is directly attached to the linking nitrogen atom.

Alternatively, R¹ is an optionally substituted heteroaryl group, and in particular is an optionally substituted monocyclic heteroaryl group such as pyridyl. Suitable values for R¹ as a heteroaryl ring include pyrimidinyl, pyridyl, pyrazolyl, pyrazinyl, thiazolyl, oxadiazolyl, isoxazolyl and thiadiazolyl.

It will be understood that when R¹ is a partially saturated bicyclic heteroaryl group, such as benzodioxolanyl, it is the aromatic ring portion of R¹ which is directly attached to the linking nitrogen atom.

Suitable values for R¹ as a bicyclic heteroaryl ring include pyrrolopyridyl, benzodioxolanyl, benzthiazolyl, benzimidazolyl and quinolyl.

More suitable values for R¹ include phenyl, naphthyl, indanyl, pyrimidinyl, pyridyl, pyrazolyl, pyrazinyl, thiazolyl, oxadiazolyl, isoxazolyl, thiadiazolyl, pyrrolopyridyl, 1,3-benzodioxan-5-yl, benzthiazolyl, benzimidazolyl and quinolyl.

In one embodiment, R¹ may not be pyrrolo[1,2-b]pyridazine.

Suitable optional substituents for R¹ include functional groups or (1-6C)alkyl groups such as methyl. Particular functional groups for substituents on R¹ include halo, —C(O)ₙR²⁰ or —OR²⁰, where R²⁰ is as defined above, and in particular is an aryl or aralkyl group.

Suitable functional groups as substituents on R¹ include halo, —OR²⁰ (wherein R²⁰ is hydrogen, phenyl or (1-4C)alkyl, optionally substituted by one or more halo, such that for example R²⁰ is difluoromethyl or trifluoromethyl, or optionally substituted by (1-4C)alkoxy), cyano, halo(1-4C)alkyl, —S(O)ₘR²⁰ (wherein R²⁰ is phenyl or (1-4C)alkyl, particularly methyl or ethyl, m is 0, 1 or 2, particularly 0 or 2), trifluoromethylthio, —NR²⁰CONR²¹R²² (wherein R²⁰, R²¹ and R²² are suitably all hydrogen), —C(O)ₙR²⁰ (wherein n is 1 or 2, particularly 2 and R²⁰ is (1-4C)alkyl or phenyl), —OSO₂R²⁰ (wherein R²⁰ is suitably (1-4C)alkyl), —SO₂NR²¹R²² (wherein R²¹ and R²² are suitably both hydrogen), —NR²¹C(O)ₙR²⁰ (wherein n is 1 or 2, particularly 1, R²¹ is suitably hydrogen and R²⁰ is suitably phenyl or (1-4C)alkyl), and —CONR²¹R²² (wherein R²¹ and R²² are suitably hydrogen).

Suitable values for Z^{a} include phenyl (optionally substituted by a functional group as hereinbefore defined, for example by —CO₂Me, or carboxy), benzyl, cyclohexyl, pyridyl, pyrimidinyl (optionally substituted by (1-4C)alkyl), triazolyl, morpholino, (2-4C)alkynyl (for example ethynyl) and (1-4C)alkyl (optionally substituted by a substituent selected from —CO₂Me, carboxy, methoxy, hydroxy and cyano).

Where R¹ is substituted by a group —X²—(CR⁵²R⁵³)_{w}-Z^{a}, suitably w is 0 or 1; Z^{a} is selected from the suitable values given above, particularly hydrocarbyl (such as optionally substituted alkyl, phenyl or benzyl) or pyridyl, and is more suitably optionally substituted phenyl; X² is suitably —SO₂—, —CO—, NHCO—, —NH—, —O—, and R⁵² and R⁵³ are suitably both hydrogen.

In another aspect, optional substituents on R¹ are 1, 2 or 3 substituents independently selected from alkyl (for example (1-6C)alkyl such as methyl or ethyl), halo, haloalkyl (such as halo(1-6C)alkyl, such as halomethyl, for example trifluoromethyl), haloalkoxy (such as halo(1-6C)alkoxy, such as halomethoxy, for example trifluoromethoxy) and cyano.

In another aspect, optional substituents on R¹ are 1, 2 or 3 substituents independently selected from fluoro, chloro, bromo, trifluoromethyl, methoxy, difluoromethoxy, trifluoromethoxy, cyano, methyl, ethyl, ethynyl, benzyloxy, 3-chlorobenzyloxy, phenoxy, 4-chlorophenoxy, phenyl, benzoyl and anilino.

In another aspect, optional substituents on R¹ are 1, 2 or 3 substituents independently selected from fluoro, cyano and trifluoromethyl. When R¹ is di- or tri-substituted, then in particular, at least 1 substituent is fluoro. When R¹ is di- or tri-substituted, preferably each substitutent is fluoro. In this aspect, particularly R¹ is phenyl.

In one aspect, R¹ is mono-substituted in the 3-position relative to the bond to NH; in another aspect R¹ is mono-substituted in the 4-position. In a further aspect R¹ is 2,4-di-substituted, 2,6-di-substituted, 3,4-di-substituted, 2,4-di-substituted, or 2,5-di-substituted by any of the possible substituents hereinbefore or hereinafter, but particularly those preferred optional substituents above, and more particularly di-halo, for example di-fluoro. In a further aspect R¹ is trisubstituted, for example 2,4,5-trisubstituted, such as 2,4,5-trihalo (for example 2, 4,5-trifluoro).

When R¹ is di- or tri-substituted, the substituents are suitably independently selected from a functional group, Z^{a} and —X²—(CR⁵²R⁵³)_{w}-Z^{a}, for example any of those values given herein for these groups. For example, R¹ may be substituted by di-halo (such as difluoro, dichloro, mono-fluoro mono-chloro and mono-chloro mono-bromo), tri-halo (such as trifluoro), mono-halo mono-alkyl (such as mono-methyl, mono-chloro), mono-halo (such as fluoro or chloro) mono-trifluoromethyl, mono-alkyl (such as methyl) mono-cyano, di-methoxy, mono-chloro mono-methoxy, di-halo mono-hydroxy (such as 2-F, 4-Cl, 5-OH), or may be for example di-halo mono —O-Z^{a} (such as —OCH₂CO₂Me). When R¹ is di-substituted, in one aspect at least one of the substituents is selected from halo, (1-4C)alkyl, (1-4C)alkoxy, trifluoromethyl and cyano. When R¹ is tri-substituted, in one aspect at least one, for example at least two, of the substituents are selected from halo, (1-4C)alkyl, (1-4C)alkoxy, trifluoromethyl and cyano.

Where R¹ is substituted by a group —X²—(CR⁵²R⁵³)_{w}-Z^{a}, a group —X²—(CR⁵²R⁵³)ₐ—X³-Z^{a} or a group —(CR⁵²R⁵³)ᵥX³-Z^{a}, R⁵² and R⁵³ are suitably hydrogen.

When R¹ is substituted by Z^{a}, wherein Z^{a} is a heterocyclyl ring, such as a morpholino ring, preferably Z^{a} is not attached to the carbon atom of R¹ which is in an ortho position to the bond to the linking nitrogen atom.

In one embodiment, Y is a direct bond.

Where Y is a group —X⁶(CR⁴⁰R⁴¹)ₜ, X⁶ is suitably oxygen and t is suitably an integer of from 2 to 6.

Alternatively, Y is a group (CH₂)ₛ or more preferably —O(CH₂)ₜ— where s is an integer of from 1 to 6 and t is an integer of from 2 to 6, and in particular s or t are 3.

When R² is unsubstituted aryl or unsubstituted cycloalkyl, Y is preferably other than a direct bond.

R² is a suitably a substituted phenyl or a substituted heteroaryl group (for example any of those heteroaryl groups listed hereinbefore). Suitable examples of R² include phenyl, pyridyl, pyrimidinyl, indanyl, cyclohexyl, piperidinyl and benzthiazolyl.

When R² is an optionally substituted cycloalkyl group, it is preferably a monocyclic group such as (3-8C)cycloalkyl or (3-6C)cycloalkyl.

When R² is a substituted group, it is suitably substituted by at least one and optionally more than one substituent group -Z, a group —X—(CR⁴²R⁴³)ᵤ-Z, a group —X—(CR⁴²R⁴³)ᵥ—X¹-Z or a group —(CR⁴²R⁴³)ᵥX¹-Z, where one or more further substituents may be selected from halo, cyano, nitro, amino, hydroxy or halo(1-6C)alkyl. Preferably R² is substituted by 1 or 2 groups independently selected from those defined hereinbefore or hereinafter, more preferably by 1 group. When R² is substituted by 2 groups, preferably one is a functional group as hereinbefore defined, such as halo, —CO₂R²⁰ (wherein R²⁰ is hydrogen, (1-4C)alkyl or allyl) or cyano, or one substituent is (1-4Calkyl.

Particular examples of groups Z or Z^{a} include groups of sub formula (x), (y) or (z).

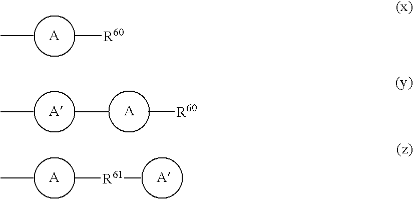

wherein each ring A or A′ is independently selected from an optionally substituted heterocyclic ring, an optionally substituted cycloalkyl ring or an optionally substituted aryl ring, each R⁶⁰ is an optionally substituted (1-6C)alkyl, an optionally substituted (2-6C)alkenyl or an optionally substituted (2-6C)alkynyl, and R⁶¹ is an optionally substituted (1-6C)alkylene, an optionally substituted (2-6C)alkenylene or an optionally substituted (2-6C)alkynylene.

Suitably optional substituents for groups A, A′, R⁶⁰ and R⁶¹ are functional groups.

A further particular example of groups Z or Z^{a} includes groups of sub formula (zz), wherein A, R⁶⁰ and R⁶¹, and suitable optional substituents therein are as defined above for sub formulae (x), (y) and (z).

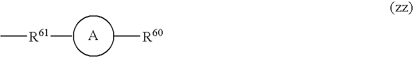

In a particularly preferred embodiment, Z is a group of sub-formula (x) above. In one embodiment, R² is a 5- or 6-membered aromatic ring of sub-structure (a):

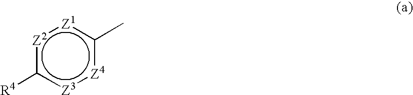

Z¹, Z², Z³ and Z⁴ are independently selected from —CH—, —CR⁶— or a heteroatom selected from —O—, —S—, —N(R⁵⁰)ᵣ—, where r is 0 or 1 depending upon the requirements of the aromatic ring, and R⁵⁰ is hydrogen or (1-6C)alkyl, and Z⁴ may additionally be a direct bond;

R⁴ is a group -Z, a group —X—(CR⁴²R⁴³)ᵤ-Z, a group —X—(CR⁴²R⁴³)ᵥ—X-Z or a group —(CR⁴²R⁴³)ᵥX¹-Z, wherein Z, X, X¹ R⁴², R⁴³, u and v are as defined above;

each R⁶ is independently selected from halo, cyano, nitro, amino, hydroxy, haloC₁₋₆alkyl, a group -Z, a group —X—(CR⁴²R⁴³)ᵤ-Z, a group —X—(CR⁴²R⁴³)ᵥ—X¹-Z or a group —(CR⁴²R⁴³)ᵥX¹-Z, wherein Z, X, X¹ R⁴², R⁴³, u and v are as defined above.

Suitably, when Z⁴ is a direct bond, one of Z¹ or Z² is a heteroatom, in particular oxygen or sulphur.

Preferably Z⁴ is other than a direct bond.

Suitably in this case, Z² and Z³ are independently selected from —CH—, —CR²⁴— or a nitrogen atom.

Suitably Z¹ is a —CH— group.

Preferred groups R⁶ are listed below as R^{6a}.

Suitably, Z¹, Z², Z³ and Z⁴ are —CH—.

Suitably R⁴² and R⁴³ are hydrogen.

Where one of Z¹ to Z⁴ is N(R⁵⁰)ᵣ, preferably it is Z² or Z³. Where one of Z¹ to Z⁴ is —CR⁶, preferably it is Z² or Z³.

In an alternative embodiment, R² is a cycloalkyl group such as cyclohexyl of sub-formula (b)

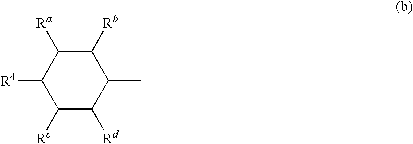

where R⁴ is as defined above, and R^{a}, R^{b}, R^{c} and R^{d} are independently selected from hydrogen or a group R⁶ as defined above.

In yet a further embodiment, R² is a bicyclic ring, which may be a bicyclic aryl ring or a bicyclic heterocyclic ring. For instance, R² comprises fused 6,6-membered rings, or fused 5,6-membered rings, one or both of said rings may be unsaturated. Examples of such rings include benzimidazole (preferably linked to the group-Y—NH— by way of the benzene ring), indanyl, indenyl. Particularly suitable bicyclic rings are partially unsaturated, such that the ring linked to the group-Y—NH— is saturated and this is fused to an aromatic ring. Particular examples of such rings are indanyl rings, such as 2-indanyl. In one embodiment, R² may not be pyrrolo[1,2-b]pyridazine.

In particular, R⁴ is a group Z.

Suitably Z is an aryl, heterocyclyl or cycloalkyl group, any of which are optionally substituted by a functional group or an (1-6C)alkyl, (2-6C)alkenyl or (2-6C)alkynyl group.

Preferably Z is substituted by a functional group or by a (1-6C)alkyl group which is substituted by a functional group. Particular examples of such functional groups include —C(O)₂R²⁰ or a carboxylic acid mimic or bioisostere thereof, —C(O)NR²¹R²² and —NR²¹C(O)ₙR²⁰, where R²⁰, R²¹ and R²² are as defined above.

In another embodiment, R² is substituted by Z and Z is an optionally substituted heterocyclyl group. Suitable examples of Z as an optionally substituted heterocyclyl group include any of the suitable values for heterocyclyl given hereinbefore and in particular include pyrrolidinyl, piperazinyl, piperidinyl, pyridyl, morpholino, thiomorpholino, homopiperazinyl, thiadiazolyl, (oxo)pyridazinyl and (oxo)thiadiazolyl.

In another embodiment, R² is substituted by Z and Z is an optionally substituted hydrocarbyl group. Suitable examples of Z as an optionally substituted hydrocarbyl group include (all optionally substituted) (1-6C)alkyl (such as (1-4C)alkyl), phenyl, cycloalkyl (such as adamantyl, cyclobutyl, cyclopentyl and cyclohexyl), cycloalkyl combined with (1-4C)alkyl (such as methylcyclohexyl, ethylcyclohexyl, isopropylcyclohexyl, cyclohexylmethyl, ethylcyclobutyl, cyclobutylmethyl and methylcyclopentyl) and phenyl combined with (1-4C)alkyl (such as benzyl and methylphenyl (such as tolyl)).

In another embodiment, R² is substituted by Z and Z is an optionally substituted combination of hydrocarbyl and heterocyclyl groups. Suitable examples of Z as an optionally substituted combination of hydrocarbyl and heterocyclyl groups include non aromatic heterocycles such as piperazinyl or piperidyl substituted by (1-4C)alkyl (for example methyl, ethyl and isopropyl), benzyl or cycloalkyl(1-4C)alkyl (for example cyclopropylmethyl); oxidised heterocycles such as oxopyridazine or oxothiadiazine substituted by one or two (1-4C)alkyl (such as methyl); aromatic heterocycles (such as pyridyl) substituted by one or two (1-4C)alkyl (such as methyl). For example pyridylmethyl (wherein the methyl may be further substituted by a functional group such as carboxy), benzylpiperazinyl, (methyl)oxopyridazinyl, (methyl)oxothiadiazolyl, (optionally carboxy substituted)methylpiperidyl, (optionally carboxy substituted)methylpiperidylmethyl, (optionally carboxy substituted)dimethylpiperidyl, (optionally carboxy substituted)ethylpiperidyl and (cyclopropylmethyl)piperazinyl.

In another embodiment R² is substituted by Z and Z is an optionally substituted combination of two heterocyclyl groups, for example pyridyl in combination with piperazinyl.

Suitable substituents on a group Z include halo, hydroxy, carboxy, —COₙR²⁰ [wherein R²⁰ is hydrogen, optionally substituted hydrocarbyl (such as (1-4C)alkyl, benzyl, phenyl, methylphenyl, phenethyl) or optionally substituted heterocyclyl (such as pyridyl) and wherein n is 1 or 2], —CONH₂, —CONHR²¹ (wherein R²¹ is selected from hydrogen, alkyl and benzyl), cyano, amino, —NHCO₂(1-4C)alkyl, and —CONR²¹R²² (wherein NR²¹R²² forms an optionally substituted heterocyclyl ring).

Suitably a ring formed by NR²¹R²² contains 0 or 1 further heteroatom selected from O, N and S and may be for example piperidinyl, piperazinyl, pyrrolidinyl or morpholino. A ring formed by NR²¹R²² may also be fused to another ring, for example thereby comprise a pyrrolidinyl ring fused with dioxolan.

In general, suitably R²⁰ is hydrogen or is selected from (all optionally substituted) (1-4C)alkyl, phenyl, pyridyl, benzyl, phenethyl, methylphenyl and allyl.

In general, R²¹ and R²² are suitably are each independently hydrogen or are selected from (optionally substituted) phenyl, (1-4C)alkyl, and benzyl.

Suitably R²⁰, R²¹ and R²² (and rings formed by NR²¹R²²) are unsubstituted or are substituted by 1 or 2 substituents. Suitable optional substitutents for R²⁰, R²¹ and R²² include halo, cyano, hydroxy, (1-4C)alkoxy, carboxy and —CO₂(1-4C)alkyl. A particular substituent for R²¹ and R²² is hydroxy. Particular substituents for rings formed by NR²¹R²² are hydroxy, carboxy and —CO₂(1-4C)alkyl.

In another embodiment R² is substituted by —X—(CR⁴²R⁴³)ᵤZ, wherein X is preferably O, —NH—, —NMe-, or —SO₂NH—, u is 0, 1 or 2, R⁴² and R⁴³ are each hydrogen and Z is selected from any of the values mentioned hereinbefore, particularly morpholino or optionally substituted phenyl (such as methoxyphenyl) or methylphenyl.

In another embodiment, R² is substituted only by a functional group as hereinbefore defined. In particular, the functional group may be selected from (1-4C)alkoxy, (1-4C)alkylthio and (1-4C)alkylsulfonyl, wherein the aforementioned (1-4C)alkyl groups may optionally be substituted by carboxy or (1-4C)alkoxycarbonyl.

As used herein, the reference to carboxylic acid mimic or bioisostere includes groups as defined in The Practice of Medicinal Chemistry, Wermuth C. G. Ed.: Academic Press: New York, 1996, p203. Particular examples of such groups include —SO₃H, S(O)₂NHR¹³, —S(O)₂NHC(O)R¹³, —CH₂S(O)₂R³, —C(O)NHS(O)₂R¹³, —C(O)NHOH, —C(O)NHCN, —CH(CF₃)OH, C(CF₃)₂OH, —P(O)(OH)₂ and groups of sub-formula (a)-(i′) below

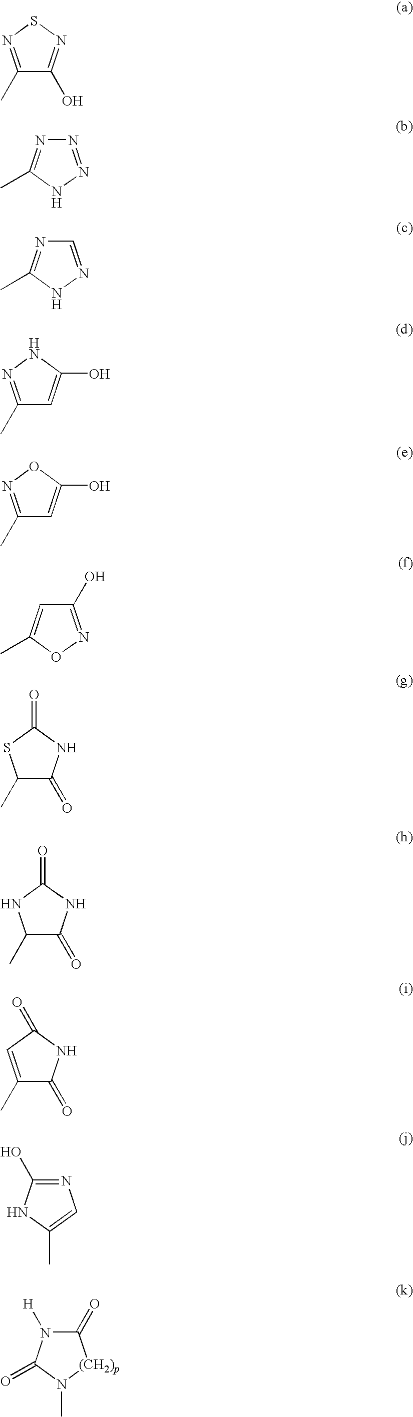

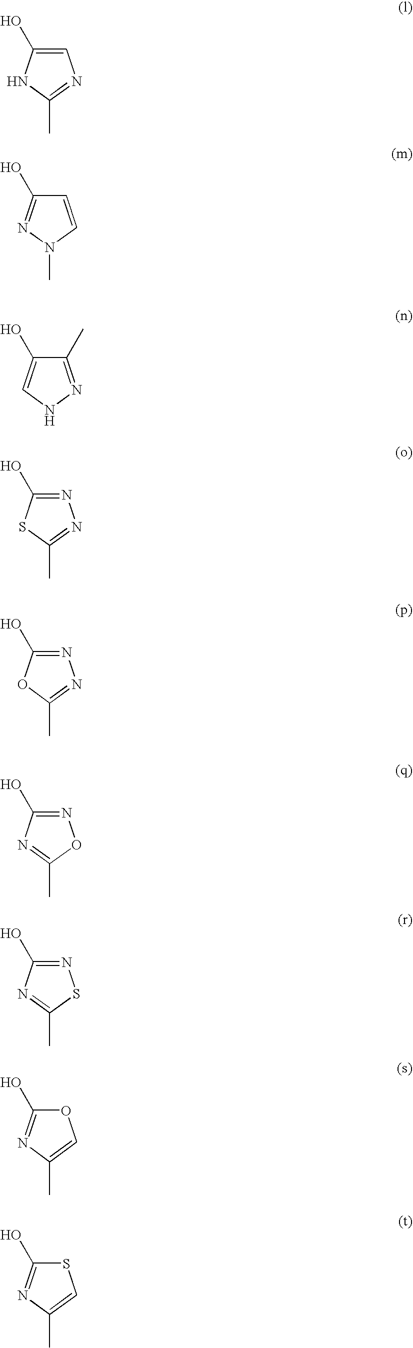

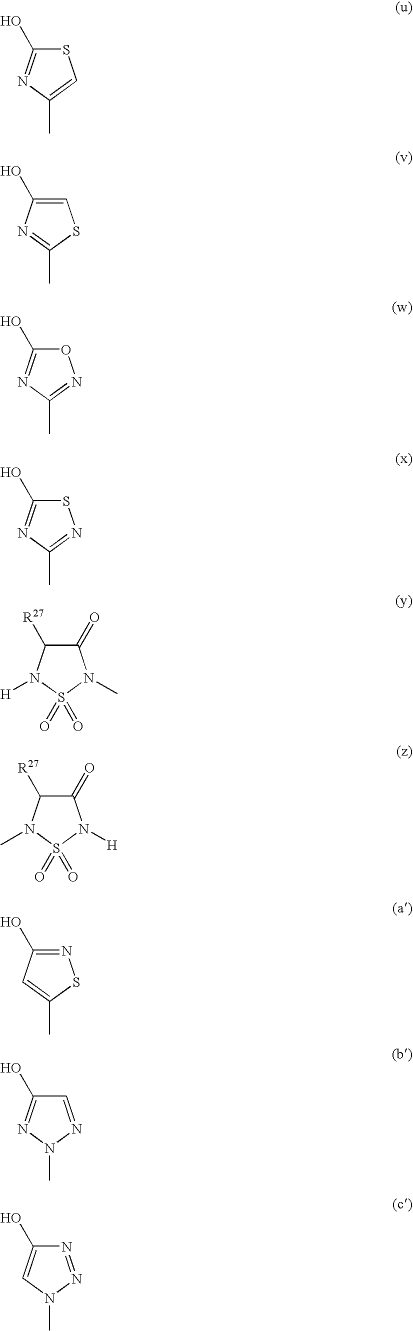

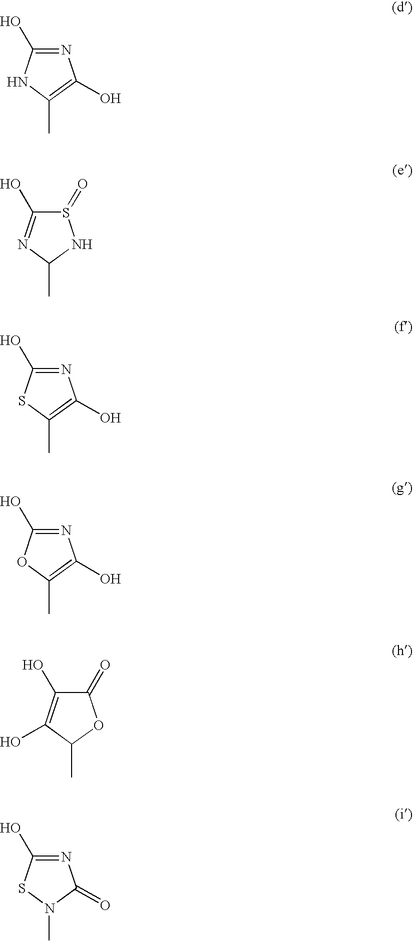

wherein R¹³ is (1-6C)alkyl, aryl or heteroaryl; and R²⁷ is hydrogen or (1-4C)alkyl. It will be understood that in the above sub-formulae (a) to (i′), keto-enol tautomerism may be possible and that the sub-formulae (a) to (i′) should be taken to encompass all tautomers thereof.

In a further aspect of the invention, there is provided a compound of formula (IZA), or a salt thereof,

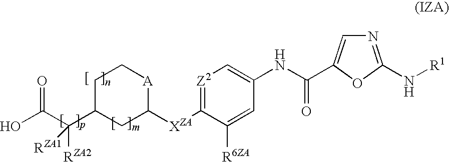

wherein R¹ is selected from phenyl (optionally substituted with 1, 2 or 3 substituents independently selected from fluoro, chloro, bromo, trifluoromethyl, methoxy, difluoromethoxy, trifluoromethoxy, cyano, methyl, ethyl, ethynyl, benzyloxy, 3-chlorobenzyloxy, phenoxy, 4-chlorophenoxy, phenyl, benzoyl and anilino), 2-pyridyl (optionally substituted by chlorophenoxy, chlorobenzyloxy or methoxyphenoxy, and/or substituted with a substituent selected from halo, trifluoromethyl, (1-4C)alkyl, (1-4C)alkoxy and cyano), 3-pyridyl (optionally substituted as for 2-pyridyl), halopyrimidinyl and trifluoromethylthiazolyl;

### Z² is N or CH;

R^{ZA1} and R^{ZA2} are each independently hydrogen or methyl;
R^{ZA3} is hydrogen or methyl;
R^{6ZA} is hydrogen, fluoro, chloro or methyl;

### A is N or CH;

X^{ZA} is a direct bond, —CH₂— or —O— (except when A is N);
m is 0, 1 or 2;
n is 0 or 1;
provided that m+n=0, 1 or 2;
p is 0 or 1.

In one aspect, for a compound of formula (IZA), wherein A is —CH—, the substituents on the ring containing A (ie the X^{ZA}-pyridyl/phenyl group and the carboxy(alkyl) group) are trans relative to each other.

In one aspect, for a compound of formula (IZA), wherein A is —CH—, the substituents on the ring containing A (ie the X^{ZA}-pyridyl/phenyl group and the carboxy(alkyl) group) are cis relative to each other.

In one embodiment, there is provided a compound of formula (IZA), or a salt thereof, wherein

R¹ is as defined hereinbefore;

### Z² is CH;

R^{ZA1} and R^{ZA2} are both hydrogen;
R^{ZA3} is hydrogen;
R^{6ZA} is hydrogen, fluoro, chloro or methyl;

### A is CH;

X^{ZA} is a direct bond, or —O—;
m is 1;
n is 1;
p is 0 or 1.

In another embodiment, there is provided a compound of formula (IZA), or a salt thereof, as defined immediately above wherein R¹ is phenyl and p is 1.

In another embodiment, there is provided a compound of formula (IZA), or a salt thereof, as defined immediately above wherein X^{ZA} is a direct bond and R^{6ZA} is hydrogen or fluoro.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

It is to be understood that where substituents contain two substituents on an alkyl chain, in which both are linked by a heteroatom (for example two alkoxy substituents), then these two substituents are not substituents on the same carbon atom of the alkyl chain.

If not stated elsewhere, suitable optional substituents for a particular group are those as stated for similar groups herein.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as a Group (I) (alkali metal) salt, a Group (II) (alkaline earth metal) salt, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions.

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DGAT1 activity and is not to be limited merely to any one tautomeric form utilised within the formulae drawings.

Pro-drugs of compounds of formula (I), or salts thereof are also within the scope of the invention.

Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Examples of such prodrugs are in vivo cleavable esters of a compound of the invention. An in vivo cleavable ester of a compound of the invention containing a carboxy group is, for example, a pharmaceutically-acceptable ester which is cleaved in the human or animal body to produce the parent acid. Suitable pharmaceutically-acceptable esters for carboxy include (1-6C)alkyl esters, for example methyl or ethyl; (1-6C)alkoxymethyl esters, for example methoxymethyl; (1-6C)alkanoyloxymethyl esters, for example pivaloyloxymethyl; phthalidyl esters; (3-8C)cycloalkoxycarbonyloxy(1-6C)alkyl esters, for example 1-cyclohexylcarbonyloxyethyl; 1,3-dioxolan-2-ylmethyl esters, for example 5-methyl-1,3-dioxolan-2-ylmethyl; (1-6C)alkoxycarbonyloxyethyl esters, for example 1-methoxycarbonyloxyethyl; aminocarbonylmethyl esters and mono- or di- N-((1-6C)alkyl) versions thereof, for example N,N-dimethylaminocarbonylmethyl esters and N-ethylaminocarbonylmethyl esters; and may be formed at any carboxy group in the compounds of this invention. An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include (1-6C)alkanoyl esters, for example acetyl esters; and benzoyl esters wherein the phenyl group may be substituted with aminomethyl or N- substituted mono- or di- (1-6C)alkyl aminomethyl, for example 4-aminomethylbenzoyl esters and 4-N,N-dimethylaminomethylbenzoyl esters.

It will be appreciated by those skilled in the art that certain compounds of formula (I) contain asymmetrically substituted carbon and/or sulfur atoms, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DGAT1 activity, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DGAT1 activity by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DGAT1 activity.

As stated before, we have discovered a range of compounds that have good DGAT1 inhibitory activity. They have good physical and/or pharmacokinetic properties in general. The following compounds possess preferred pharmaceutical and/or physical and/or pharmacokinetic properties.

In one embodiment of the invention there are provided compounds of formula (I), in an alternative embodiment there are provided pharmaceutically-acceptable salts of compounds of formula (I). In a further embodiment, there are provided pro-drugs, particularly in-vivo cleavable esters, of compounds of formula (I). In a further embodiment, there are provided pharmaceutically-acceptable salts of pro-drugs of compounds of formula (I). Reference herein to a compound of formula (I) should in general be taken to apply also to compounds of formula (IZA).

Further preferred compounds of the invention are each of the Examples, each of which provides a further independent aspect of the invention. In further aspects, the present invention also comprises any two or more compounds of the Examples.

### Process

A compound of formula (I) and its pharmaceutically-acceptable salts may be prepared by any process known to be applicable to the preparation of chemically related compounds. Such processes, when used to prepare a compound of the formula (I), or a pharmaceutically-acceptable salt thereof, are provided as a further feature of the invention.

In a further aspect the present invention also provides that the compounds of the formula (I) and salts thereof, can be prepared by a process a) to f) as follows (wherein all variables are as hereinbefore defined for a compound of formula (I) unless otherwise stated):

a) reaction of a compound of formula (I) to form another compound of formula (I);

b) reaction of an amine of formula (2) with a carboxylic acid compound of formula (3);

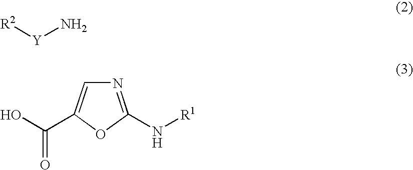

c) when R² is substituted by piperazinyl, by reaction of the piperazine nitrogen with R⁵-LG wherein LG is a suitable leaving group such as halo, and R⁵ is hydrocarbyl or a suitable functional group such as acyl, for example:

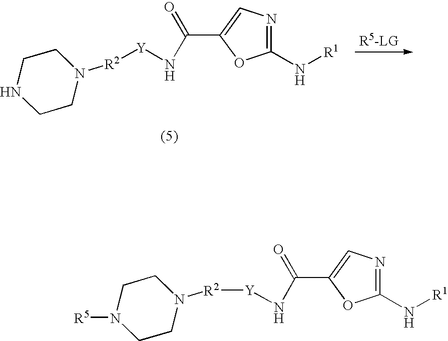

d) when R² is aryl and is substituted by aryl, by transition metal catalysed aromatic substitution (with NH protection where necessary), for example:

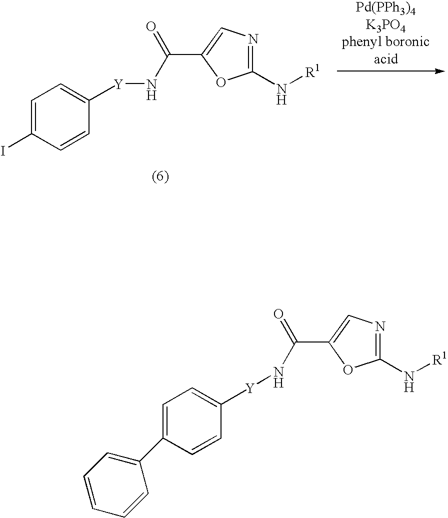

e) when R² is substituted by piperazinyl, by reductive alkylation of the piperazine nitrogen with R⁵—CHO (wherein R⁵ is for example hydrocarbyl), for example:

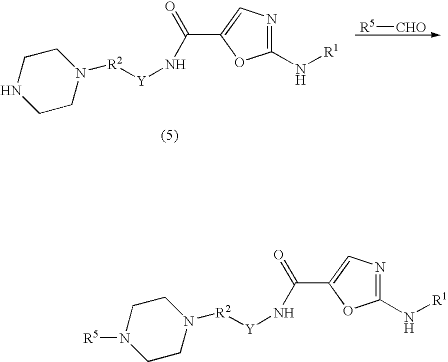

f) reaction of halogenated (for example with iodo) R² with an amide of formula (7) followed by subsequent removal of protecting group P¹, wherein P¹ is for example benzyl or trimethylsilylethoxymethyl (SEM), for example;

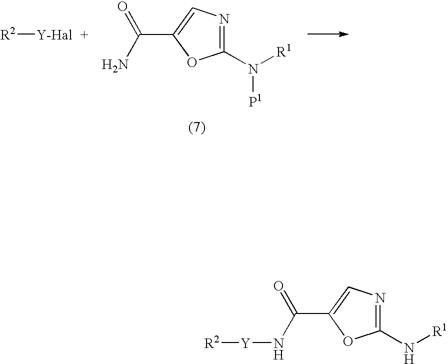

wherein Hal is halogen;

and thereafter if necessary, removing any protecting groups, and/or forming a salt thereof.

In the above schemes, R¹, R², Y and R⁵ are as defined above. It will be understood that, where Y is a direct bond, processes a,b and f apply to compounds of formula (IZA).

### Process a)

Examples of conversions of a compound of formula (I) into another compound of Formula (I), well known to those skilled in the art, include functional group interconversions such as hydrolysis (in particular ester hydrolysis), oxidation or reduction (such as the reduction of an acid to an alcohol, or removal of an N protecting group), and/or further functionalisation by standard reactions such as amide or metal-catalysed coupling, or nucleophilic displacement reactions.

### Process b)

Compounds of formula (2) where Y is not a direct bond or where R² is not aromatic, may be made by application of standard synthetic methods well known in the art. For example, reductive alkylation of ammonia (or a suitable amine such as a benzylamine or N,N-dibenzylamine) with a ketone or aldehyde R²Y═O (followed by deprotection as appropriate) provides R²—Y—NH₂. Alternatively, alkylation of an amine or amine equivalent (such as a Gabriel reagent or a guanidine) with a halide R²—Y—X (where X is a halide) (followed by N-deprotection or hydrolysis as appropriate) provides the required compounds of formula (2).

Compounds of formula (2) for other definitions of Y or R² may be made by metal catalysed couplings or nucleophilic displacement reactions among other methods. In particular, compounds of formula (2) may be prepared by reduction of a compound of formula (2A).

R²—Y—NO₂ (2A)

Compounds of formula (2A) may be made by metal catalysed couplings or nucleophilic displacement reactions depending upon the nature of the R² group and Y. For example, production of a compound of formula (2A) may be represented as follows:

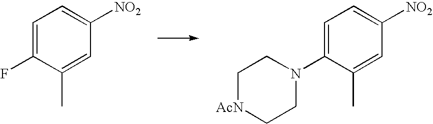

Examples of process (b) where Y is a direct bond are shown in Schemes 1 to 3 (wherein R⁶ represents optional substituent on R²):

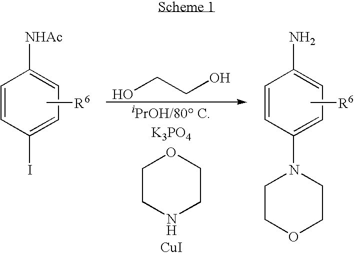

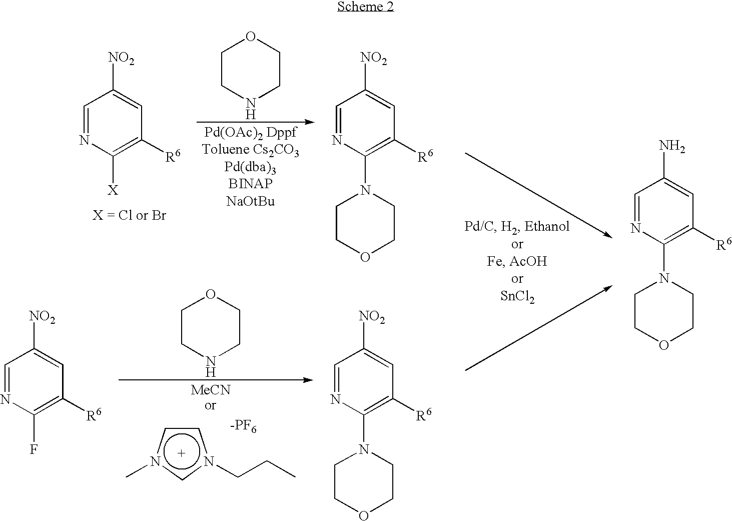

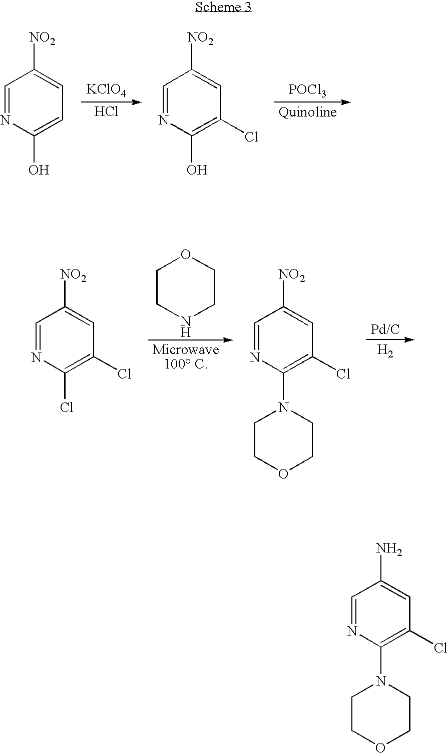

Certain compounds of formula (2) may also have chiral centres or can exist in different isomeric forms such as cis/trans isomers, and may be prepared as individual isomers, as illustrated below in Scheme 4.

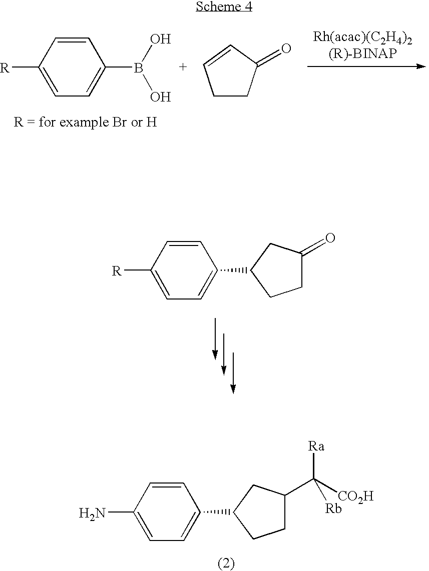

The process illustrated in Scheme 4 may also be used with cyclohexenone as a starting material. The opposite stereochemistry may be obtained by using known alternative chiral catalysts and/or chiral ligands. Elaboration of the bicyclic ketone intermediate may be carried out by processes known in the art, for example by Wittig or enolate/enol ether chemistry, optionally followed by functionalisation (such as alkylation) and functional group interconversion as desired to give the compound of formula (2) (wherein Ra and Rb may each for example be hydrogen or (optionally substituted) alkyl groups). Mixtures of diastereoisomers may be separated by standard procedures.

S_{N}Ar chemistry may be used (under conditions well known in the art) to make certain compounds of formula (2), as illustrated in Scheme 5 (in which R is for example an alkyl group, X is for example Br or Cl, n is for example 0 to 4, group A may be a (hetero)aryl ring, a saturated ring or an alkyl chain, and R⁶ represents optional substituent on R²).

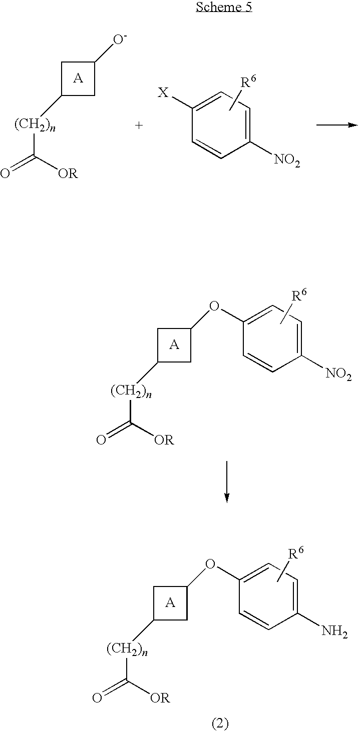

Compounds of formula (3) may be made by alkaline hydrolysis of ester (8). Ester (8) may be made by reaction of a compound of formula (9) with a compound of formula (10) for example at elevated temperatures (such as 150° C.). Alternatively, a compound of formula (9) may be reacted with a compound of formula (11) to form the oxazole ring, and then with a compound of formula R¹—X, where X is a leaving group such as halo, for example using transition metal catalysis, to give a compound of formula (8).

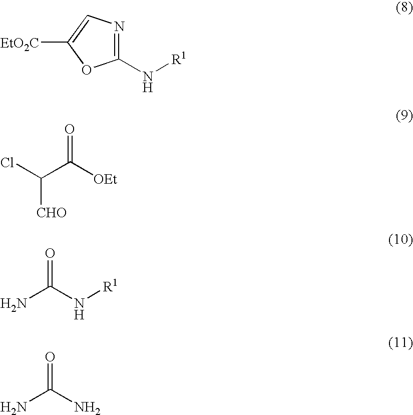

Compounds of formula (2) may be coupled with compounds of formula (3) under standard conditions for formation of amide bonds. For example using an appropriate coupling reaction, such as a carbodiimide coupling reaction performed with EDAC, optionally in the presence of DMAP, in a suitable solvent such as DCM, chloroform or DMF at room temperature.

For compounds of formula (2) other than when R² is aromatic and Y is a direct bond (ie other than compounds such as anilino compounds), an ester derivative of formula (8) (or equivalent) may be used instead of the compound of formula (3) to couple with the compound of formula (2). Such a reaction may be carried out by any method known in the art such as by heating (thermally or by microwave) in a suitable solvent.

### Process c)

Compounds of formula (5) can be reacted with an acid chloride or sulfonyl chloride in the presence of a base such as triethylamine or pyridine in a suitable solvent such as DMF.

### Process d)

Compounds of formula (6) can be reacted with aryl boronic acids in the presence of a suitable catalyst such as tetrakis(triphenyl phosphine)palladium(0) and a suitable base such as potassium phosphate in a suitable solvent such as DME-water (2:1) under microwave heating at 0 to 110° C.

### Process e)

Compounds of formula (5) can be reacted with aldehydes in the presence of a suitable acid such as acetic acid, and a reducing agent such as sodium borohydride in a suitable solvent such as DCM.

### Process f)

Compounds of formula (7) can be reacted with arylbromides, aryliodides, aryltrifluoromethanesulfonates, heteroarylbromides or heteroaryliodides in the presence of a suitable catalyst such as copper(i) iodide, a suitable diamine ligand such as trans-N,N′-dimethyl-1,2-cyclohexyldiamine and a suitable base such as potassium phosphate in a suitable solvent such as DMF or dioxane heating at 80-110° C.

It will be appreciated that certain of the various ring substituents in the compounds of the present invention, for example Z, Za, and/or R³, may be introduced by standard aromatic substitution reactions or generated by conventional functional group modifications either prior to or immediately following the processes mentioned above, and as such are included in the process aspect of the invention. Such reactions may convert one compound of the formula (I) into another compound of the formula (I). Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkanesulfinyl or alkanesulfonyl.

If not commercially available, the necessary starting materials for the procedures such as those described above may be made by procedures which are selected from standard organic chemical techniques, techniques which are analogous to the synthesis of known, structurally similar compounds, techniques which are described or illustrated in the references given above, or techniques which are analogous to the above described procedure or the procedures described in the examples. The reader is further referred to Advanced Organic Chemistry, 5^{th} Edition, by Jerry March and Michael Smith, published by John Wiley & Sons 2001, for general guidance on reaction conditions and reagents.

It will be appreciated that some intermediates to compounds of the formula (I) are also novel and these are provided as separate independent aspects of the invention.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl or SEM may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the examples herein, to obtain necessary starting materials, and products.

The removal of any protecting groups and the formation of a pharmaceutically-acceptable salt are within the skill of an ordinary organic chemist using standard techniques. Furthermore, details on the these steps has been provided hereinbefore.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further aspect of the invention there is provided a pharmaceutical composition which comprises a compound of formula (I) and (IZA) as defined hereinbefore or a pharmaceutically-acceptable salt thereof, in association with a pharmaceutically-acceptable excipient or carrier.

Certain intermediates used in the processes described above are novel, and these form a further aspect of the invention. In particular, compounds of formula (4) form a further aspect of the invention.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

According to a further aspect of the present invention there is provided a compound of formula (I) or a pharmaceutically acceptable salt thereof as defined hereinbefore for use in a method of treatment of the human or animal body by therapy.

Reference herein to a compound of formula (I) should be understood to refer equally to compounds of formula (I) and (IZA).

We have found that compounds of the present invention inhibit DGAT1 activity and are therefore of interest for their blood glucose-lowering effects.

A further feature of the present invention is a compound of formula (I) or a pharmaceutically-acceptable salt thereof for use as a medicament.

Conveniently this is a compound of formula (I), or a pharmaceutically-acceptable salt thereof, for use as a medicament for producing an inhibition of DGAT1 activity in a warm-blooded animal such as a human being.

Particularly this is a compound of formula (I), or a pharmaceutically-acceptable salt thereof, for use as a medicament for treating diabetes mellitus and/or obesity in a warm-blooded animal such as a human being.

Thus according to a further aspect of the invention there is provided the use of a compound of formula (I), or a pharmaceutically-acceptable salt thereof in the manufacture of a medicament for use in the production of an inhibition of DGAT1 activity in a warm-blooded animal such as a human being.

Thus according to a further aspect of the invention there is provided the use of a compound of formula (I), or a pharmaceutically-acceptable salt thereof in the manufacture of a medicament for use in the treatment of diabetes mellitus and/or obesity in a warm-blooded animal such as a human being.

According to a further aspect of the invention there is provided a pharmaceutical composition which comprises a compound of formula (I) as defined hereinbefore or a pharmaceutically-acceptable salt thereof, in association with a pharmaceutically-acceptable excipient or carrier for use in producing an inhibition of DGAT1 activity in an warm-blooded animal, such as a human being.

According to a further aspect of the invention there is provided a pharmaceutical composition which comprises a compound of formula (I) as defined hereinbefore or a pharmaceutically-acceptable salt thereof, in association with a pharmaceutically-acceptable excipient or carrier for use in the treatment of diabetes mellitus and/or obesity in an warm-blooded animal, such as a human being.

According to a further feature of the invention there is provided a method for producing an inhibition of DGAT1 activity in a warm-blooded animal, such as a human being, in need of such treatment which comprises administering to said animal an effective amount of a compound of formula (I) or a pharmaceutically-acceptable salt thereof as defined hereinbefore.

According to a further feature of the invention there is provided a method of treating diabetes mellitus and/or obesity in a warm-blooded animal, such as a human being, in need of such treatment which comprises administering to said animal an effective amount of a compound of formula (I) or a pharmaceutically-acceptable salt thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

As stated above compounds defined in the present invention are of interest for their ability to inhibit the activity of DGAT1. A compound of the invention may therefore be useful for the prevention, delay or treatment of a range of disease states including diabetes mellitus, more specifically type 2 diabetes mellitus (T2DM) and complications arising there from (for example retinopathy, neuropathy and nephropathy), impaired glucose tolerance (IGT), conditions of impaired fasting glucose, metabolic acidosis, ketosis, dysmetabolic syndrome, arthritis, osteoporosis, obesity and obesity related disorders, (which include peripheral vascular disease, (including intermittent claudication), cardiac failure and certain cardiac myopathies, myocardial ischaemia, cerebral ischaemia and reperfusion, hyperlipidaemias, atherosclerosis, infertility and polycystic ovary syndrome); the compounds of the invention may also be useful for muscle weakness, diseases of the skin such as acne, Alzheimer's disease, various immunomodulatory diseases (such as psoriasis), HIV infection, inflammatory bowel syndrome and inflammatory bowel disease such as Crohn's disease and ulcerative colitis.

In particular, the compounds of the present invention are of interest for the prevention, delay or treatment of diabetes mellitus and/or obesity and/or obesity related disorders. In one aspect, the compounds of the invention are used for prevention, delay or treatment of diabetes mellitus. In another aspect, the compounds of the invention are used for prevention, delay or treatment of obesity. In a further aspect, the compounds of the invention are used for prevention, delay or treatment of obesity related disorders.

The inhibition of DGAT1 activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example such conjoint treatment may be beneficial in the treatment of metabolic syndrome [defined as abdominal obesity (as measured by waist circumference against ethnic and gender specific cut-points) plus any two of the following: hypertriglyceridemia (>150 mg/dl; 1.7 mmol/l); low HDLc (<40 mg/dl or <1.03 mmol/l for men and <50 mg/dl or 1.29 mmol/l for women) or on treatment for low HDL (high density lipoprotein); hypertension (SBP≧130 mmHg DBP≧85 mmHg) or on treatment for hypertension; and hyperglycemia (fasting plasma glucose ≧100 mg/dl or 5.6 mmol/l or impaired glucose tolerance or pre-existing diabetes mellitus)—International Diabetes Federation & input from IAS/NCEP].

Such conjoint treatments may include the following main categories:
1) Anti-obesity therapies such as those that cause weight loss by effects on food intake, nutrient absorption or energy expenditure, such as orlistat, sibutramine and the like.
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1, 6 bisphosphatase inhibitors, glycogen phopsphorylase inhibitors, glycogen synthase kinase inhibitors, glucokinase activators);
6) Agents designed to reduce the absorption of glucose from the intestine (for example a carbose);
7) Agents that prevent the reabsorption of glucose by the kidney (SGLT inhibitors);
8) Agents designed to treat the complications of prolonged hyperglycaemia (for example aldose reductase inhibitors);
9) Anti-dyslipidaemia agents such as, HMG-CoA reductase inhibitors (eg statins); PPARα-agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
10) Antihypertensive agents such as, β-blockers (eg atenolol, inderal); ACE inhibitors (eg lisinopril); Calcium antagonists (eg. nifedipine); Angiotensin receptor antagonists (eg candesartan), α antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
12) Agents which antagonise the actions of glucagon; and
13) Anti-inflammatory agents, such as non-steroidal anti-inflammatory drugs (eg. aspirin) and steroidal anti-inflammatory agents (eg. cortisone).

In addition to their use in therapeutic medicine, compounds of formula (I) and their pharmaceutically-acceptable salts are also useful as pharmacological tools in the development and standardisation of in vitro and in vivo test systems for the evaluation of the effects of inhibitors of DGAT1 activity in laboratory animals such as cats, dogs, rabbits, monkeys, rats and mice, as part of the search for new therapeutic agents.

As indicated above, all of the compounds, and their corresponding pharmaceutically-acceptable salts, are useful in inhibiting DGAT1. The ability of the compounds of formula (I), and their corresponding pharmaceutically-acceptable acid addition salts, to inhibit DGAT1 may be demonstrated employing the following enzyme assay:

### Human Enzyme Assay

The in vitro assay to identify DGAT1 inhibitors uses human DGAT1 expressed in insect cell membranes as the enzyme source (Proc. Natl. Acad. Sci. 1998, 95, 13018-13023). Briefly, sf9 cells were infected with recombinant baculovirus containing human DGAT1 coding sequences and harvested after 48 h. Cells were lysed by sonication and membranes isolated by centrifuging at 28000 rpm for 1 h at 4° C. on a 41% sucrose gradient. The membrane fraction at the interphase was collected, washed, and stored in liquid nitrogen.

DGAT1 activity was assayed by a modification of the method described by Coleman (Methods in Enzymology 1992, 209, 98-102). Compound at 1-10 μM was incubated with 0.4 μg membrane protein, 5 mM MgCl₂, and 100 μM 1,2 dioleoyl-sn-glycerol in a total assay volume of 200 μl in plastic tubes. The reaction was started by adding ¹⁴C oleoyl coenzyme A (30 μM final concentration) and incubated at room temperature for 30 minutes. The reaction was stopped by adding 1.5 mL 2-propanol:heptane:water (80:20:2). Radioactive triolein product was separated into the organic phase by adding 1 mL heptane and 0.5 mL 0.1 M carbonate buffer pH 9.5. DGAT1 activity was quantified by counting aliquots of the upper heptane layer by liquid scintillography.

Using this assay the compounds generally show activity with IC₅₀<20 μM, particularly <10 μM, more particularly <1 μM. Example 1 showed an IC₅₀=0.1 μM.

The ability of the compounds of formula (I), and their corresponding pharmaceutically-acceptable acid salts, to inhibit DGAT1 may further be demonstrated employing the following whole cell assays 1) and 2):

### 1) Measurement of Triglyceride Synthesis in 3T3 Cells

Mouse adipocyte 3T3 cells were cultured to confluency in 6 well plates in new born calf serum containing media. Differentiation of the cells was induced by incubating in medium containing 10% foetal calf serum, 1 μg/mL insulin, 0.25 μM dexamethasone and 0.5 mM isobutylmethyl xanthine. After 48 h the cells were maintained in medium containing 10% foetal calf serum and 1 μg/mL insulin for a further 4-6 days. For the experiment, the medium was changed to serum-free medium and the cells pre-incubated with compound solubilised in DMSO (final concentration 0.1%) for 30 minutes. De novo lipogenesis was measured by the addition of 0.25 mM sodium acetate plus 1 μCi/mL ¹⁴C-sodium acetate to each well for a further 2 h (J. Biol. Chem., 1976, 251, 6462-6464). The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (1997). Incorporation of radiolabel into the triglyceride fraction was analysed using a Radiomatic Flo-one Detector (Packard) connected to the HPLC machine.

### 2) Measurement of Triglyceride Synthesis in MCF7 Cells

Human mammary epithelial (MCF7) cells were cultured to confluency in 6 well plates in foetal calf serum containing media. For the experiment, the medium was changed to serum-free medium and the cells pre-incubated with compound solubilised in DMSO (final concentration 0.1%) for 30 minutes. De novo lipogenesis was measured by the addition of 50 μM sodium acetate plus 3 μCi/mL ¹⁴C-sodium acetate to each well for a further 3 h (J. Biol. Chem., 1976, 251, 6462-6464). The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (J. Chromat. B, 1997, 703, 7-14). Incorporation of radiolabel into the triglyceride fraction was analysed using a Radiomatic Flo-one Detector (Packard) connected to the HPLC machine.

In the above other pharmaceutical composition, process, method, use and medicament manufacture features, the alternative and preferred embodiments of the compounds of the invention described herein also apply.

### EXAMPLES

The invention will now be illustrated by the following Examples in which, unless stated otherwise:

(i) temperatures are given in degrees Celsius (° C.); operations were carried out at room or ambient temperature, that is, at a temperature in the range of 18-25° C. and under an atmosphere of an inert gas such as argon;
(ii) organic solutions were dried over anhydrous magnesium sulfate; evaporation of solvent was carried out using a rotary evaporator under reduced pressure (600-4000 Pa; 4.5-30 mmHg) with a bath temperature of up to 60° C.;
(iii) chromatography means flash chromatography on silica gel; where a Biotage cartridge is referred to this means a cartridge containing KP-SIL™ silica, 60 Å, particle size 32-63 mM, supplied by Biotage, a division of Dyax Corp., 1500 Avon Street Extended, Charlottesville, Va. 22902, USA;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data (¹H) is in the form of delta values for major diagnostic protons, given in parts per million (ppm) relative to tetramethylsilane (TMS), determined at 300 or 400 MHz (unless otherwise stated) using perdeuterio dimethyl sulfoxide (DMSO-d₆) as solvent, unless otherwise stated; peak multiplicities are shown thus: s, singlet; d, doublet; dd, doublet of doublets; dt, doublet of triplets; dm, doublet of multiplets; t, triplet, q, quartet; m, multiplet; br, broad;
(vii) chemical symbols have their usual meanings; SI units and symbols are used;
(viii) solvent ratios are given in volume: volume (v/v) terms;
(ix) mass spectra (MS) (loop) were recorded on a Micromass Platform LC equipped with HP 1100 detector; unless otherwise stated the mass ion quoted is (MH⁺);
(x) LCMS (liquid chromatography-mass spectrometry) were recorded on a system comprising Waters 2790 LC equipped with a Waters 996 Photodiode array detector and Micromass ZMD MS, using a Phenomenex® Gemini 5u C18 110A 50×2 mm column and eluting with a flow rate of 1.1 ml/min with 5% (Water/Acetonitrile (1:1)+1% formic acid) and a gradient increasing from 0-95% of acetonitrile over the first 4 minutes, the balance (95-0%) being water and where HPLC Retention Times are reported these are in minutes in this system unless otherwise stated; unless otherwise stated the mass ion quoted is (MH⁺);
(xi) where phase separation cartridges are stated then ISOLUTE Phase Separator 70 ml columns, supplied by Argonaut Technologies, New Road, Hengoed, Mid Glamorgan, CF82 8AU, United Kingdom, were used;
(xii) where a SiliCycle cartridge is referred to this means a cartridge containing Ultra Pure Silica Gel particle size 230-400 mesh, 40-63 um pore size, supplied by SiliCycle Chemical Division, 1200 Ave St-Jean-Baptiste, Suite 114, Quebec City, Quebec, G2E,5E8, CANADA;
(xiii) where an Isco Companion is referred to then a Combiflash companion chromatography instrument, supplied by ISOC Inc. Address Teledyne ISOC Inc, 4700 Superior Street, Lincoln, Nebr. 68504, USA, was used;
(xiv) where a microwave is referred to this means a Biotage Initiator sixty or Smith Creator microwave, supplied by Biotage, a division of Dyax Corp., 1500 Avon Street Extended, Charlottesville, Va. 22902, USA;
(xv) where GCMS is referred to then a Gas Chromatography—Mass Spectrometry analysis was carried out on a QP-2010 GC-MS system fitted with an AOC 20i autosampler and controlled by ‘GCMS solutions’ software, version 2.0, supplied by Shimadzu, Milton Keynes, MK12 5RE, UK; the GC column was a DB-5MS of length 25 m, 0.32 mm i.d. with a film thickness of 0.52 μm supplied by J & W Scientific, Folsom, Calif., USA;
(xvi) where a centrifuge is referred to this means a Genevac EZ-2plus, supplied by Genevac Limited, The Soveriegn Centre, Farthing Road, Ipswich, IPI 5AP, UK;
(xvii) where chiral chromatography is referred to this is carried generally out using a 20 μm Merck 50 mm Chiralpak AD column, (Chiral Stationary Phase supplied by Chiral Technologies Europe, Parc d'Innovation, Bd. Gonthier d'Andemach, 67404 Illkirch Cedex, France), using MeCN/2-propanol/AcOH (90/10/0.1) as eluent, flow rate 80 mL/min, wavelength 300 nm, using a Gilson prep HPLC instrument (200 ml heads);
(xviii) melting points were determined using a Buchi 530 apparatus and are uncorrected;
(xix) Reverse phase preparative HPLC separations were run on standard Gilson™ HPLC equipment using a 150×21.2 mm Phenomenex Luna 10 micron C18(2) 100A column, and a standard gradient elution method (5-95% acetonitrile gradient with water as co-solvent and 0.2% trifluoroacetic acid as modifier, 12.5 min gradient with a 2.5 min hold at 95% acetonitrile) run on Unipoint software.
(xx) The following abbreviations may be used below or in the process section hereinbefore:
Et₂O or ether diethyl ether
DMF dimethylformamide
DCM dichloromethane
DME 1,2-dimethoxyethane
MeOH methanol
EtOH ethanol
H₂O water
TFA trifluoroacetic acid
THF tetrahydrofuran
DMSO dimethylsulfoxide
HOBt 1-hydroxybenzotriazole
EDCI (EDAC) 1-ethyl-3-(3-dimethylaminopropyl)carbodi-imide hydrochloride
DIPEA diisopropylethylamine
DEAD diethyl azodicarboxylate
EtOAc ethyl acetate
NaHCO₃ sodium bicarbonate/sodium hydrogencarbonate
K₃PO₄ potassium phosphate
PS polymer supported
BINAP 2,2′-bis(diphenylphosphino)-1,1′ binaphthyl
Dppf 1,1′-bis(diphenylphosphino)ferrocene
dba dibenzylidineacetone
PS-CDI polymer supported carbonyldiimidazole
CH₃CN or MeCN acetonitrile
h hour
min minute
HATU O-(7-Azabenzotriazol-1-yl)-N,N,N′,N′-tetramethyluronium hexofluorophosphate
NaOH sodium hydroxide
AcOH acetic acid
DMA dimethyl acetamide
nBuLi n-butyl lithium
MgSO₄ magnesium sulfate
Na₂SO₄ sodium sulfate
CDCl₃ deutero chloroform
CD₃OD per-deuterated methanol
Boc tert-butoxycarbonyl
HCl hydrochloric acid

All final compound names were derived using ACD NAME computer package.

### Example 1

### [4-(4-{[(2-Anilino-1,3-oxazol-5-yl)carbonyl]amino}phenyl)cyclohexyl]acetic acid

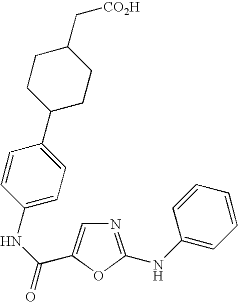

Lithium hydroxide monohydrate (16 mg, 0.38 mmol) was added to a stirred solution of ethyl [4-(4-{[(2-anilino-1,3-oxazol-5-yl)carbonyl]amino}phenyl)cyclohexyl]acetate (Intermediate 1, 93 mg, 0.21 mmol) and the reaction mixture was stirred at 85° C. for 5 h. The mixture was cooled to ambient temperature and acidified with a 2N aqueous solution of HCl. The resulting precipitate was filtered to give the title compound as a white solid (60 mg, 68%).

¹H NMR (400 MHz, DMSO) δ1.07-1.23 (m, 2H), 1.44-1.59 (m, 2H), 1.76-1.96 (m, 5H), 2.13 (d, 2H), 2.44-2.53 (m, 1H), 7.02-7.09 (m, 1H), 7.23-7.31 (m, 2H), 7.35-7.43 (m, 2H), 7.61-7.78 (m, 4H), 7.88-7.94 (m, 1H), 10.03 (s, 1H), 10.21 (s, 1H), 10.81 (s, 1H); MS m/e MH⁺ 420.

### Intermediate 1

### Ethyl [4-(4-{[(2-anilino-1,3-oxazol-5-yl)carbonyl]amino}phenyl)-cyclohexyl]acetate

### i) Ethyl 2-anilino-1,3-oxazole-5-carboxylate

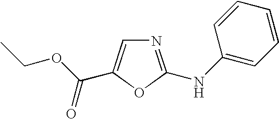

Ethyl 2-chloro-3-oxopropanoate (Ref: Heterocycles, 1991, 32(4), 693, 520 mg, 3.47 mmol) was added in one portion to N-phenylurea (566 mg, 4.16 mmol) in water (1 mL) and the reaction mixture was heated to 150° C. for 5 mins in the microwave. The resulting precipitate was washed with water and then triturated with EtOAc. The filtrate was concentrated in vacuo and purified by reverse phase preparative HPLC to give the product as a white solid (130 mg, 16%).

¹H NMR δ1.26 (t, 3H), 4.21 (q, 2H), 7.07 (t, 1H), 7.33 (t, 2H), 7.46 (d, 2H), 8.19 (s, 1H), 9.42 (s, 1H); MS m/e MH⁺ 233.

### ii) 2-Anilino-1,3-oxazole-5-carboxylic acid

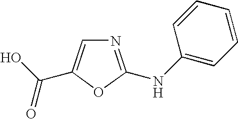

Lithium hydroxide monohydrate (167 mg, 3.97 mmol) was added in one portion to a stirred solution of ethyl 2-anilino-1,3-oxazole-5-carboxylate (184 mg, 0.79 mmol) in THF (2 mL) and water (2 mL) and the reaction mixture was stirred at 70° C. for 1 h. The mixture was acidified with a 2N aqueous solution of HCl and the resulting suspension was filtered to give the crude product as a white solid (57 mg, 35%).

¹H NMR δ 6.95-7.06 (m, 1H), 7.27-7.39 (m, 2H), 7.60 (d, 2H), 7.72 (s, 1H), 10.71 (s, 1H), 12.98 (s, 1H); MS m/e MH⁺ 205.

### iii) Ethyl [4-(4-{[(2-anilino-1,3-oxazol-5-yl)carbonyl]amino}phenyl)cyclohexyl]acetate

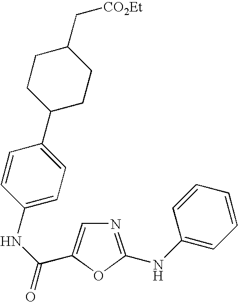

EDCI (175 mg, 0.91 mmol) was added in one portion to a stirred solution of 2-anilino-1,3-oxazole-5-carboxylic acid (93 mg, 0.46 mmol), HOBt (123 mg, 0.91 mmol), ethyl [4-(4-aminophenyl)cyclohexyl]acetate (178 mg, 0.68 mmol) and DIPEA (238 μL, 1.37 mmol) in DMF (5 mL). The reaction mixture was stirred at ambient temperature for 16 h under a nitrogen atmosphere. Water (10 mL) was added and the resulting precipitate was filtered and washed with diethyl ether (10 mL) to give the title compound (Intermediate 1) as a white solid (120 mg, 59%).

¹H NMR δ1.20 (t, 3H), 1.38-1.53 (m, 2H), 1.54-1.69 (m, 2H), 1.70-1.87 (m, 5H), 2.21 (d, 2H), 2.36-2.46 (m, 1H), 4.07 (q, 2H), 7.00 (t, 1H), 7.18 (d, 2H), 7.34 (t, 2H), 7.53-7.69 (m, 4H), 7.87 (s, 1H), 9.93 (s, 1H), 10.65 (s, 1H); MS m/e MH⁺ 448.

### Intermediate 2

### Ethyl [4-(4-aminophenyl)cyclohexyl]acetate

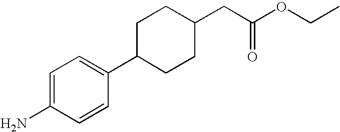

A mixture of nitric acid (65%, 3.5 mL) and sulphuric acid (95%, 4.4 mL) in carbon tetrachloride (5 mL) was added dropwise over 10 mins to a stirred solution of ethyl 2-(4-phenylcyclohexyl)acetate (described in WO2004/047755 compound 3, 4.43 g, 18.0 mmol) in carbon tetrachloride (10 mL) at 5° C. The solution was warmed to ambient temperature and stirred for 16 h. Water (50 mL), cooled to 5° C., and DCM (40 mL) were added to the reaction mixture. The layers were separated and the aqueous layer was extracted with DCM (40 mL). The organic layers were combined, washed with brine (50 mL), dried (MgSO₄) and concentrated in vacuo to leave an oil. The oil was purified by flash chromatography, using a gradient of 0% to 20% EtOAc in isohexane as eluent, to give the intermediate nitrophenyl compound as a crude solid (4.68 g) that was used with no further purification. 10% palladium on carbon (500 mg) was added to a solution of the crude nitrophenyl intermediate (4.66 g) in EtOAc (30 ml) at ambient temperature. The reaction mixture was stirred at ambient temperature for 16 h under a hydrogen atmosphere. The reaction mixture was filtered and concentrated in vacuo to leave a solid, which was purified by flash chromatography, using a gradient of 0 to 60% EtOAc in isohexane as eluent, to give the title compound as a solid (2.20 g, 47%).

¹H NMR (CDCl₃) δ 1.07-1.18 (m, 2H), 1.27 (t, 2H), 1.39-1.70 (m, 4H), 1.78-1.94 (m, 2H), 2.18-2.55 (m, 3H), 3.52 (broad s, 2H), 4.13 (quartet, 2H), 6.63 (d, 2H), 7.01 (d, 2H); MS m/e MH⁺ 262.

1. A compound of formula (I) 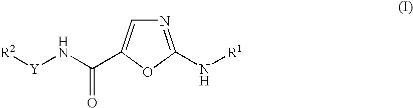
or a salt thereof, wherein:
R¹ is an optionally substituted aryl or optionally substituted heteroaryl group, wherein the optional substituents are one or more selected from -Z^{a}, —X²—(CR⁵²R⁵³)_{w}-Z^{a}, —X²—(CR⁵²R⁵³)ₐ—X³-Z^{a}, —(CR⁵²R⁵³)ₐX³-Z^{a} or a functional group other than —X²—(CR⁵²R⁵³)_{w}-Z^{a} or —X² (CR⁵²R⁵³)ₐ—X³-Z^{a};
Y is a direct bond, (CR⁴⁰R⁴¹), or —X⁶ (CR⁴⁰R⁴¹)ₜ—;
each R⁴⁰ and R⁴¹ is independently selected from hydrogen, (1-4C)alkyl, hydroxy, halo, halo(1-4C)alkyl, amino, cyano, (1-4C)alkoxy, (1-4C)haloalkoxy or ((1-3C)alkyl)CONH—;
s is an integer of from 1 to 6;
t is an integer of from 1 to 6,
provided that the X⁶ atom of —X⁶(CR⁴⁰R⁴¹)ₙ— is attached to R² and that a single sp³ hybridised carbon atom does not carry two or more bonds to a heteroatom unless the heteratom is a halo;
R² is an optionally substituted aryl, an optionally substituted cycloalkyl or an optionally substituted heterocyclic group, wherein the optional substitutents are one or more selected from -Z, —X—(CR⁴²R⁴³)ᵤ-Z, —X—(CR⁴²R⁴³)—X-Z, —(CR⁴²R⁴³)ᵥX¹-Z or a functional group other than —X—(CR⁴²R⁴³)ᵤ-Z or —X—(CR⁴²R⁴³)ᵥ—X¹-Z;
Z and Z^{a} are independently selected from a hydrocarbyl group or a heterocyclic group or a combination thereof, wherein Z and Z^{a} are optionally substituted on any available atom by one or more functional groups, or by —X⁷—(CR⁶²R⁶³)_{b}R⁶⁴;
X, X¹, X², X³, X⁶ and X⁷ are linking groups independently selected from —C(O)ₓ—, —O—, —S(O)_{y}—, —NR⁴⁴—, —C(O)NR⁴⁴—, —OC(O)NR⁴⁴—, —CH═NO—, —NR⁴⁴C(O)ₓ— —NR⁴⁴CONR⁴⁵—, —S(O)₂NR⁴⁴— and —NR⁴⁴S(O)₂—;
x is 1 or 2;
y is 0, 1 or 2;
R⁴⁴ and R⁴⁵ are independently selected from hydrogen and (1-6C)alkyl;
u and w are independently 0 to 6;
v, a and b are independently 1 to 6;
each R⁴², R⁴³, R⁵², R⁵³, R⁶² and R⁶³ is independently selected from hydrogen, (1-4C)alkyl, hydroxy, halo, halo(1-4C)alkyl, amino, cyano, (1-4C)alkoxy, (1-4C)haloalkoxy, ((1-3C)alkyl)CONH—, carboxy, —SO₃H, —S(O)₂NHR¹³, —S(O)₂NHC(O)R¹³, —CH₂S(O)₂R¹³, —C(O)NHS(O)₂R¹³, —C(O)NHOH, —C(O)NHCN, —CH(CF₃)OH, C(CF₃)₂OH, —P(O)(OH)₂ and a 5-membered heterocyclic ring selected from the group consisting of 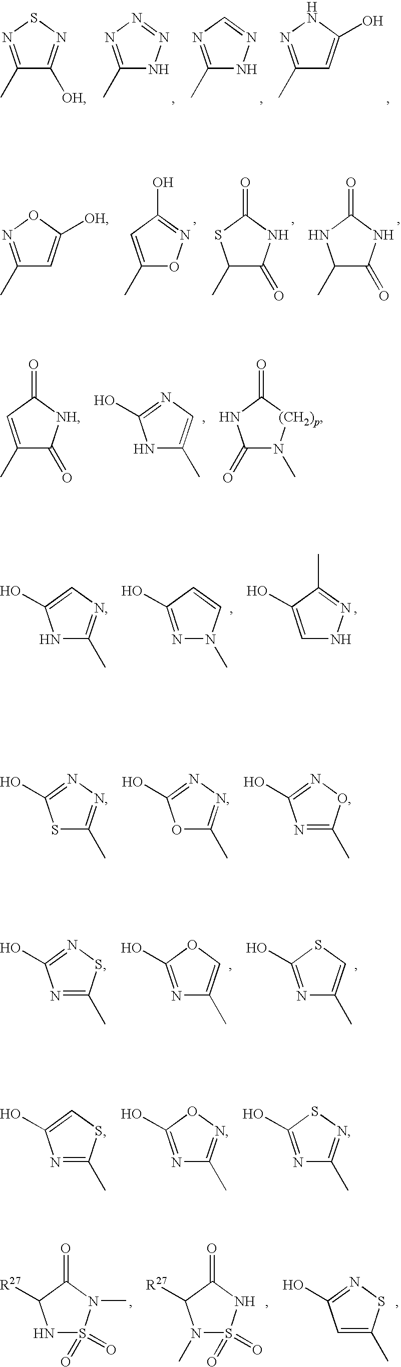 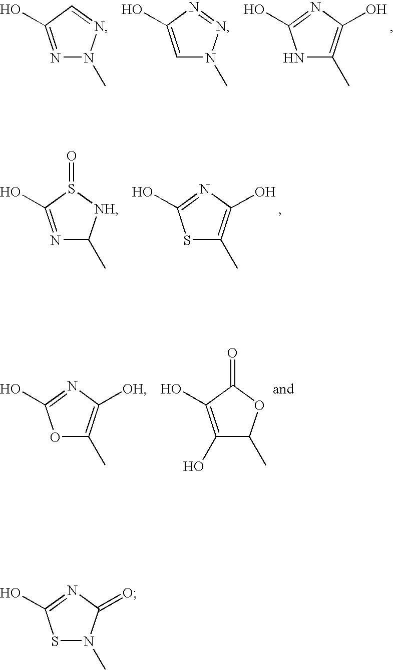
R¹³ is (1-6C)alkyl, aryl or heteroaryl;
R²⁷ is hydrogen or (1-4C)alkyl;
R⁶⁴ is a functional group,
wherein the functional group is selected from carboxy, halo, halo(1-6C)alkyl, cyano, nitro, —C(O)ₙR²⁰—R²⁰, —S(O)ₘR²⁰—OS(O)₂R²⁰, —NR²¹R²², —C(O)NR²¹R²², —OC(O)NR²¹R²², —CH═NOR²⁰, —NR²¹C(O)ₙR²⁰, —NR²⁰CONR²¹R²², —N═CR²¹R²², —S(O)₂NR²¹R²², —NR²¹S(O)₂R²², —SO₃H, —S(O)₂NHR¹³—S(O)₂NHC(O)R¹³, —CH₂S(O)₂R¹³—C(O)NHS(O)₂R¹³, —C(O)NHOH, —C(O)NHCN, —CH(CF₃)OH, —C(CF₃)₂OH, —P(O)(OH)₂ and the 5-membered heterocyclic ring as defined above;
R²⁰, R²¹ and R²² are independently selected from hydro en, optionally substituted hydrocarbyl and optionally substituted heterocyclyl, or R²¹ and R²² together with the nitrogen atom to which they are attached form an optionally substituted ring having 3 to 10 atoms, which optionally contains further heteroatoms such as S(O)ₘ, oxygen and nitrogen;
m is independently 0, 1 or 2; and
n is 1 or 2.

2. The compound according to claim 1 which is [4-(4-{[(2-Anilino-1,3-oxazol-5-yl)carbonyl]amino}phenyl)cyclohexyl]acetic acid or a salt thereof.

3. (canceled)

4. A method for producing an inhibition of DGAT1 activity in a warm-blooded animal in need of such treatment, comprising administering to the animal an effective amount of a compound of formula (I) as claimed in claim 1 or a pharmaceutically-acceptable salt thereof.

5. A method of treating diabetes mellitus and/or obesity in a warm-blooded animal in need of such treatment, comprising administering to the animal an effective amount of a compound of formula (I) as claimed in claim 1 or a pharmaceutically-acceptable salt thereof.

6-7. (canceled)

8. A pharmaceutical composition comprising a compound of formula (I) as claimed in claim 1 or a pharmaceutically-acceptable salt thereof, in association with a pharmaceutically-acceptable excipient or carrier.

9. A process for preparing a compound according to claim 1, comprising one of the following steps, wherein all variables are as hereinbefore defined for a compound of formula (I) unless otherwise stated:
a) reacting a compound of formula (I) to form another compound of formula (I);
b) reacting an amine of formula (2) with a carboxylic acid compound of formula (3) 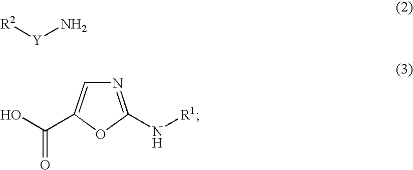
c) when R² is substituted by piperazinyl, reacting the piperazine nitrogen of formula (5) with R⁵-LG wherein LG is a suitable leaving group and R⁵ is hydrocarbyl, acyl or another suitable functional group: 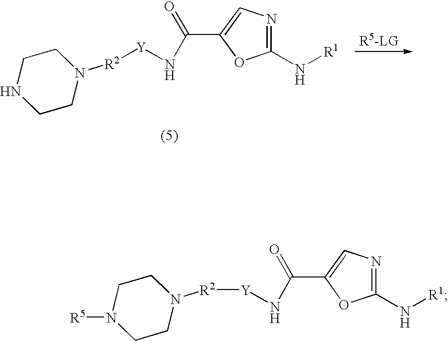
d) when R² is aryl and is substituted by aryl, transition metal catalysed aromatic substitution, optionally with NH protection, of formula (6) where an exemplary aryl is shown as phenyl: 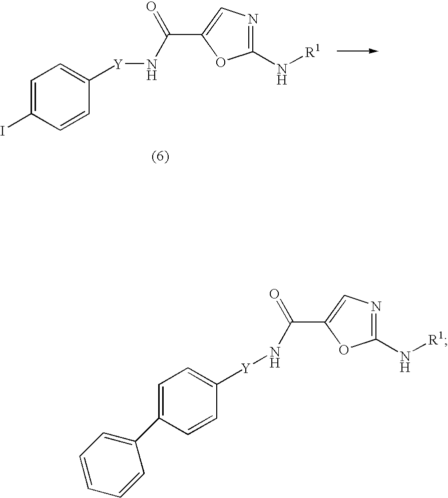
e) when R² is substituted by piperazinyl, reductive alkylation of the piperazine nitrogen of formula (5) with R⁵—CHO wherein R⁵ is hydrocarbyl or another suitable functional group: 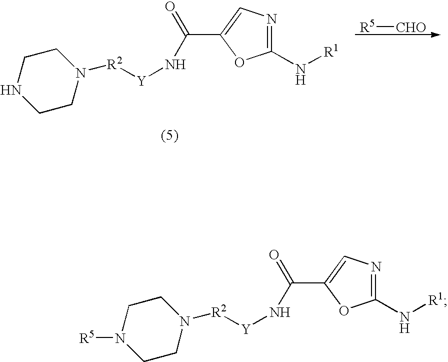
f) reacting halogenated R² with an amide of formula (7) followed by subsequent removal of protecting group P¹, wherein P¹ is benzyl, trimethylsilylethoxymethyl (SEM) or another protecting group: 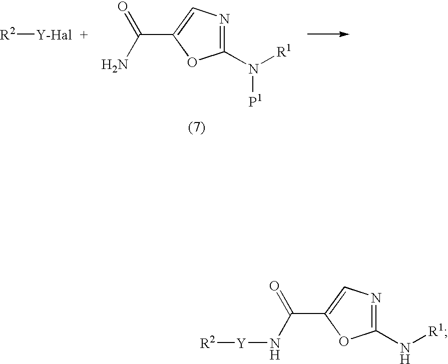
wherein Hal is halogen;
and optionally thereafter, removing any protecting groups, and/or forming a salt thereof.

10. A process for preparing a compound of formula (3) 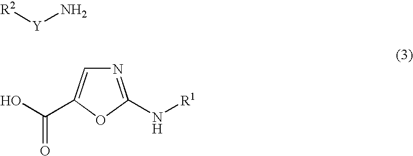
comprising reacting a compound of formula (9) with a compound of formula (11) at elevated temperature, wherein R¹ is as defined in claim 1 [chemical structure]
